(19) United States
(12) Patent Application Publication
(10) Pub. No.: US 2002/0136705 A1
(43) Pub. Date: Sep. 26, 2002

(54) PORCINE SPINAL CORD CELLS AND THEIR USE IN SPINAL CORD REPAIR

(76) Inventor: JONATHAN DINSMORE, BROOKLINE, MA (US)

Correspondence Address: AMY E. MANDRAGOURAS, LAHIVE AND COCKFIELD, 28 STATE STRTEET, BOSTON, MA, 02109

(21) Appl. No.: 09/163,272

(22) Filed: Sep. 29, 1998

Related U.S. Application Data
(60) Provisional application No. 60/091,193, filed on Jun. 30, 1998.

(51) Int. Cl. A61K 35/30; C12N 5/06

(52) U.S. Cl. 424/93.1; 424/570; 435/325

(57) ABSTRACT

Porcine spinal cord cells and methods for using the cells to treat spinal cord damage due to neurodegeneration resulting from spinal cord injury and neurodegenerative disorders are described. The porcine spinal cord cells are preferably embryonic spinal cord cells obtained from select gestational days. The porcine spinal cord cells can be modified to be suitable for transplantation into a xenogeneic subject, such as a human. For example, the porcine spinal cord cells can be modified such that an antigen (e.g., an MHC class I antigen) on the cell surface which is capable of stimulating an immune response against the cell in a xenogeneic subject is altered (e.g., by contact with an anti-MHC class I antibody, or a fragment or derivative thereof) to inhibit rejection of the cell when introduced into the subject. In one embodiment, the porcine spinal cord cells are obtained from a pig which is essentially free from organisms or substances which are capable of transmitting infection or disease to the recipient subject. The porcine spinal cord cells of the present invention can be used to treat spinal cord damage due to neurodegeneration in the spinal cord of a xenogeneic subject (e.g., a human having spinal cord injury, amyotrophic lateral sclerosis or multiple sclerosis) by introducing the cells into the spinal cord of the subject.

### RELATED APPLICATIONS

This application claims priority to U.S. provisional application Application No. 60/091,193, filed on Jun. 30, 1998, hereby incorporated in its entirety by this reference.

### BACKGROUND OF THE INVENTION

It has been estimated that the yearly incidence of traumatic spinal cord injury is 10,000 people in the U.S. per year. The U.S. prevalence is approximately 200,000 people. The total cost, including medical, rehabilitative and indirect costs, for spinal cord injury is approximately $10 billion dollars per year (Johnson et al. (1996) Spinal Cord 34:470-480). Novel therapeutic approaches which promise to alleviate sensorimotor dysfunction not only have personal benefit for individuals who have received a debilitating spinal cord injury, but also can reduce health care expenditures.

For many years, the general consensus which pervaded the field of spinal cord injury repair was that the adult spinal cord did not have regenerative capacity. This prevailing sentiment has been replaced by the belief that given the proper environment for regrowth, neurons within the central nervous system can regenerate and form functional connections (Reier (1992) J. Neurotrauma 9:S223-S248; Bregman et al. (1997) Adv. Neurology 72:257-275).

In addition, there are many neurological disorders, including amyotrophic lateral sclerosis (ALS), that are characterized by the selective degeneration of different subsets of neurons, resulting in unique clinical syndromes. ALS selectively affects motor neurons, which develop cytoskeletal pathological features and eventually die. Classic ALS has a worldwide prevalence of more than 5 out of 100,000 people. (Mohr and Gautier, eds., Guide to Clinical Neurology, (1995) Chuchill & Livingston, Inc., New York, N.Y., pp. 3-5). This disorder usually begins later in life and is inexorably progressive as patients become increasingly incapacitated.

One promising therapeutic approach to repair the lost and degenerating neuronal circuitry following damage to the spinal cord is the implantation of human fetal spinal cord tissue into the injury site. Fetal neuronal cells have the potential to re-populate the injury site secrete growth factors, and provide a local environment into which host neurons can grow. While this is not a new approach (the first experiments using fetal rat spinal cord allotransplants were conducted in the early 80's), the rationale for renewed consideration of cellular therapy for the treatment of spinal damage, e.g., spinal cord injury, is the recent progress of cellular based treatments to reduce the consequences of such injury. (See Bregman et al. (1997) Adv. Neurology 72:257-275; Bernstein et al. (1995) Paraplegia 33:250-253). However, there is a scarcity of human donors available to provide spinal cord tissue for transplantation. Thus, a need exists for an alternate source of spinal cord cells for transplantation.

### SUMMARY OF THE INVENTION

The present invention is based, at least in part, on the discovery that porcine spinal cord cells and, preferably, porcine embryonic spinal cord cells isolated during certain stages of gestational development, when transplanted into the spinal cord of a xenogeneic subject, populate areas of lost or degenerating neurons.

Accordingly, the instant invention pertains to a composition comprising a porcine spinal cord cell or an isolated population of porcine spinal cord cells suitable for transplantation into a xenogeneic subject, particularly a human subject. Preferably, these spinal cord cells are obtained from embryonic pigs during selected stages of gestational development. For example, it has been determined that embryonic spinal cord cells obtained from an embryonic pig between about days 20 and 40, preferably between about days 20 and 30, and more preferably between about days 25 and 29 of gestation are suitable for transplantation into xenogeneic subjects, particularly human subjects. The population of porcine spinal cord cells of this invention can include at least two different cell types selected from, for example, motor neurons, interneurons, sensory neurons, glial cells (e.g., oligodendrocytes and astrocytes) and precursors of these cells. Another aspect of the invention includes an isolated porcine spinal cord cell which, when transplanted into a xenogeneic subject, treats spinal cord damage. The spinal cord cells can treat areas of spinal damage by preventing or reducing further degeneration, promoting neural circuitry regrowth and/or promoting or enhancing locomotor recovery.

In one aspect of the invention, the porcine spinal cord cell, in unmodified form, has at least one antigen on the cell surface which is capable of stimulating an immune response against the cell in a xenogeneic subject, for example, a human. The antigen on the surface of the porcine spinal cord cell is altered to inhibit rejection of the cell when introduced into a xenogeneic subject. In one embodiment, the cell surface antigen which is altered is an MHC class I antigen. This MHC class I antigen can be contacted, prior to transplantation into a xenogeneic subject with at least one MHC class I antibody, or a fragment or derivative thereof, which binds to the MHC class I antigen on the cell surface but does not activate complement or induce lysis of the cell. One example of an MHC class I antibody is an MHC class I F(ab′)₂ fragment, such as an MHC class I F(ab′)₂ fragment of a monoclonal antibody PT85.

A still further aspect of the invention pertains to a porcine spinal cord cell or a population of spinal cord cells isolated from a pig predetermined to be essentially free from organisms which are capable of transmitting infection or disease to a xenogeneic recipient, e.g., a human, of the cells.

Categories of pathogens from which the swine are free can include zoonotic, cross-placental and neurotropic organisms. Within each of these categories, the organisms can be parasites, bacteria, mycoplasma, and/or viruses. In one embodiment, the pig from which the spinal cord cells are isolated is free of one or more of the following organisms: Toxoplasma, eperythrozoon, brucella, listeria, mycobacterium TB, leptospirillum, hemophilus suis, M. Hypopneumonia, porcine respiratory reproductive syndrome, rabies, pseudorabies, parvovirus, encephalomyocarditis virus, swine vesicular disease, teschen (Porcine polio virus), hemagglutinating encephalomyocarditis, suipoxvirus, swine influenza type A, adenovirus, transmissible gastroenteritis virus, bovine viral diarrhea, and vesicular stomatitis virus. The cells can be modified as described above to inhibit rejection of the cell upon introduction into a xenogeneic subject. Methods for isolating porcine spinal cord cells from such a pig, in which the pig is tested for the presence or absence of organisms which are capable of transmitting infection or disease to a recipient of the cells, and spinal cord cells isolated, are also within the scope of this invention.

The porcine spinal cord cells of the invention can be grown as a cell culture in a medium suitable to support the growth of the cells. Such cell cultures can also include at least one agent or factor such as a neurotrophic factor which promotes cell growth. Examples of neurotrophic factors include glial cell line-derived growth factor, brain-derived neurotrophic factor, ciliary neurotrophic factor, platelet-derived growth factor, neural growth factor, midkine, insulin-like growth factor I and II, insulin, fibroblast growth factor, neurotrophin-3, neurotrophin 4/5 and transforming growth factor β. In addition, the porcine spinal cord cells can be inserted into a delivery device, e.g., a syringe or micropipette, which facilitates the introduction of the cells into a subject. Preferred spinal cord cell types, cell ages, and cells sources are described above. Additionally, methods for treating spinal cord damage in a subject, e.g., a human, by introducing porcine spinal cord cells or spinal cord stem cells isolated from such a pig into the subject are contemplated by the present invention.

This invention also provides methods for treating spinal cord damage in the spinal cord of a xenogeneic subject by administering the composition of the porcine spinal cord cells of the invention, e.g., modified or unmodified porcine spinal cord cells, into a damaged spinal area. Spinal cord damage can be the result of insult and/or degeneration caused by injury to the spinal cord or neurodegenerative disorders. Transplantation of the porcine spinal cord cells can be accompanied by administration of least one agent or factor selected from the group consisting of neurotrophic factors and anti-inflammatory agents (e.g., steriods, e.g., methylprednisolone) as described herein as well as an immunosuppressive agent, e.g., cyclosporine A, FK506, RS-61443, or a T cell antibody, to the subject.

In one embodiment, spinal cord damage in a xenogeneic subject is treated by administration of a composition comprising porcine spinal cord cells (e.g., embryonic spinal cord cells) which reduce or prevent neurodegeneration upon administration of the composition into the subject. Thus, the method can be used to treat neurodegeneration associated with neurodegenerative disorders, such as amyotrophic lateral sclerosis (ALS) and multiple sclerosis (MS). It is preferred that porcine spinal cord cells are obtained from the spinal cord of an embryonic pig (e.g., embryonic days 20 to 40, or between about embryonic days 20 to 30, or between about embryonic days 25 to 29) are transplanted into the site of spinal damage. In another embodiment, spinal cord damage in a xenogeneic subject is treated by transplantation of porcine spinal cord cells (e.g., embryonic spinal cord cells) which enhance or promote recovery of the damaged area (e.g., locomotor recovery, promotion of neural sparing, production of neurotrophic factors). Thus, the methods can be used to treat insult and/or neurodegeneration associated with spinal cord injury.

### BRIEF DESCRIPTION OF THE DRAWINGS

FIG. 1 is showing the time course of behavioral recovery after spinal cord injury in the open field, grid walking, and contact placing test in response to porcine fetal spinal cord implanted cells. After hemisection, animals were transplanted with gelfoam impregnated with fetal spinal cord cells and were either immunosuppressed with cyclosporin (filled squares, n=8), or non-immunosuppressed (open squares, n=3). The ordinate is mean percent recovery above that observed for control animals which after hemisection were treated with saline-soaked gelfoam. Error bars are not shown.

FIG. 2 is showing the effect of various cell doses on open field, grid walking and contact placing tests in hemisected immunosuppressed porcine spinal cord recipient rats The ordinate is mean percent recovery above that observed for control animals which after hemisection were treated with saline-soaked gelfoam. Error bars are not shown.

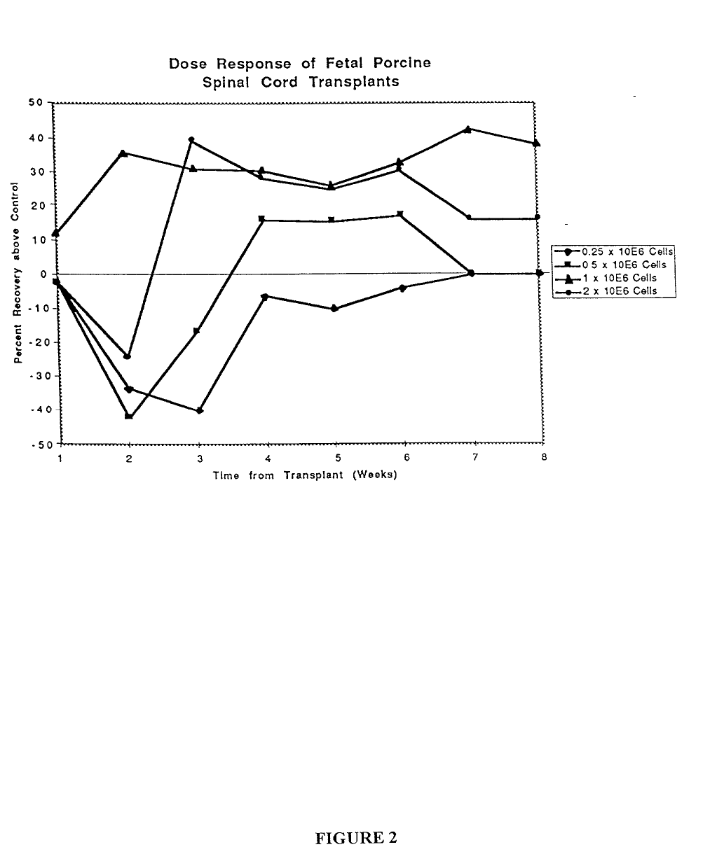

FIG. 3 is illustrating the time course of locomotor activity in an open field. A maximum score of 21 corresponds to consistent plantar stepping and coordinated gait with consistent trunk stability, while the minimum score of 0 equals no observable hindlimb movement of the injured hindpaw.

FIG. 4 is illustrating the results of the time course of the grasping reflex tests monitored by examining the rat's ability to grasp a wire mesh screen (60 cm×45 cm). The ability to grasp onto 5 open squares (1 cm×1 cm) was assessed. A score of 5 corresponds to 5 successful grasps, while a score of 0 demonstrates a rat's inability to grasp while transversing all 5 open squares. All rats received a score of 5 prior to surgical intervention.

FIG. 5 is illustrating the results of the time course of the placing reflex test monitored by examining the rat's ability to place the injured hindlimb when the limb is brought into contact with the cage surface. The ability to place the foot was assessed for 5 trials. A score of 5 corresponds to 5 successful placements, while a score of 0 demonstrates a rat's inability to place for all 5 open trials. All rats received a score of 5 prior to surgical intervention.

FIG. 6 is representing the time course of the righting reflex test. The righting reflex is assessed by the ability of the rat to “right” (land on all four paws instantaneously) when released 5 cm above the surface ventral side up. A score of 2 corresponds to instantaneous righting, a score of 1 is received when righting occurs, but it is not immediate, and 0 corresponds to the inability to right. All rats received a score of 2 prior to surgical intervention.

FIG. 7 is representing the time course demonstrating the ability of rats to transverse an elevated 4.7 cm wooden beam. A score of 5 denotes correct placement and coordinated movement of both feet, 4 corresponds to proper foot placement of the injured hindpaw without coordinated locomotion, 3 equals occasional foot placement and stepping, 2 corresponds to dragging the injured foot when walking on the beam, 1 denotes the ability to place the ipsilateral foot on the beam but no stepping occurs, and 0 is the inability to balance. All rats received a score of 5 prior to surgical intervention.

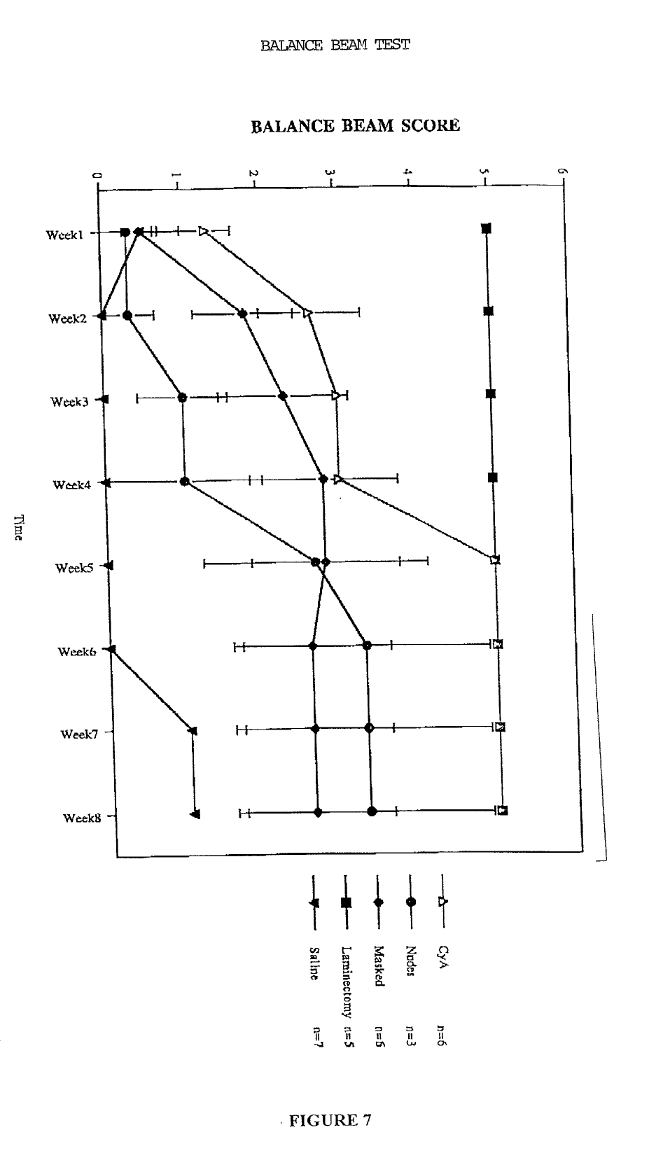

FIG. 8 is representing the time course of the inclined plane test. The maximum angle that a rat could maintain balance for 5 seconds ipsilateral to injury was determined using the inclined plane test.

### DETAILED DESCRIPTION OF THE INVENTION

### I. Isolated Cells and Cell Populations of the Invention

A. Modified Porcine Spinal Cord Cells and an Isolated Population of Modified Porcine Spinal Cord Cells

This invention features porcine spinal cord cells suitable for introduction into a xenogeneic recipient, particularly a human subject. As used herein the phrase “spinal cord cell” includes both neural cells (i.e., neurons, e.g., motor neurons, interneurons and sensory neurons) and their precursors and glial cells (e.g., macroglia such as oligodendrocytes and astrocytes) and their precursors. The terms “precursor”, “progenitor”, and “stem cell” are used interchangeably herein and refer to cells which are pluripotent, i.e., are capable of developing into many different cell types, including spinal cord cells. Such stem cells can be used as sources of the porcine spinal cord cells of the invention, i.e., the spinal cord cells of the invention can be derived from such stem cells. As used herein, the term “derived” refers to cells which develop or differentiate from or have as ancestors pluripotent stem cells.

Spinal cord cells of the invention can be obtained from any location in the pig spinal cord or peripheral nervous system. Preferably, the cells are obtained from the spinal cord. In unmodified form, the porcine spinal cord cell has at least one antigen on the cell surface which is capable of stimulating an immune response against the cell in a xenogeneic subject. To inhibit rejection of the cell when introduced into the xenogeneic subject, the antigen on the cell surface is altered prior to transplantation. In an unaltered state, the antigen on the cell surface stimulates an immune response against the cell when the cell is administered to a subject (also referred to herein as recipient or recipient subject). By altering the antigen, the normal immunological recognition of the porcine spinal cord cell by the immune system cells of the recipient is disrupted and additionally, “abnormal” immunological recognition of this altered form of the antigen can lead to porcine spinal cord cell-specific long term unresponsiveness in the recipient. It is likely that alteration of an antigen on the porcine spinal cord cell prior to introducing the cell into a subject interferes with the initial phase of recognition of the porcine spinal cord cell by the cells of the host's immune system subsequent to administration of the cell. Furthermore, alteration of the antigen may induce immunological nonresponsiveness or tolerance, thereby preventing the induction of the effector phases of an immune response (e.g., cytotoxic T cell generation, antibody production etc.) which are ultimately responsible for rejection of foreign cells in a normal immune response. As used herein, the term “altered” encompasses changes that are made to at least one porcine spinal cord cell antigen(s) which reduces the immunogenicity of the antigen to thereby interfere with immunological recognition of the antigen(s) by the recipient's immune system.

Antigens to be altered according to the current invention include antigens on a porcine spinal cord cell which can interact with an immune cell in a xenogeneic recipient subject and thereby stimulate a specific immune response against the porcine spinal cord cell in the recipient. The interaction between the antigen and the immune cell may be an indirect interaction (e.g., mediated by soluble factors which induce a response in the immune cell, e.g., humoral mediated) or, preferably, is a direct interaction between the antigen and a molecule present on the surface of the immune cell (i.e., cell-cell mediated). As used herein, the term “immune cell” is intended to include a cell which plays a role in specific immunity (e.g., is involved in an immune response) or plays a role in natural immunity. Examples of immune cells include all distinct classes of lymphocytes (T lymphocytes, such as helper T cells and cytotoxic T cells, B lymphocytes, and natural killer cells), monocytes, macrophages, other antigen presenting cells, dendritic cells, and leukocytes (e.g., neutrophils, eosinophils, and basophils). In a preferred embodiment, the antigen is one which interacts with a T lymphocyte in the recipient (e.g., the antigen normally binds to a receptor on the surface of a T lymphocyte).

In one embodiment, the antigen on the porcine spinal cord cell to be altered is an MHC class I antigen. Alternatively, an adhesion molecule on the cell surface, such as NCAM-1 or ICAM-1, can be altered. An antigen which stimulates a cellular immune response against the cell, such as an MHC class I antigen, can be altered prior to transplantation by contacting the cell with a molecule which binds to the antigen. A preferred molecule for binding to the antigen is an antibody, or fragment thereof (e.g., an anti-MHC class I antibody, or fragment thereof, an anti-ICAM- 1 antibody or fragment thereof, an anti-LFA-3 antibody or fragment thereof, or an anti-β₂ microglobulin antibody or fragment thereof). A preferred antibody fragment is an F(ab′)₂ fragment. Polyclonal or, more preferably, monoclonal antibodies can be used. Other molecules which can be used to alter an antigen (e.g., an MHC class I antigen) include peptides and small organic molecules which bind to the antigen. Furthermore, two or more different epitopes on the same or different antigens on the cell surface can be altered. A particularly preferred monoclonal antibody for alteration of MHC class I antigens on porcine spinal cord cells is PT85 (commercially available from Veterinary Medicine Research Development, Pullman WA). PT85 can be used alone to alter MHC class I antigens or, if each antibody is specific for a different epitope, PT85 can be used in combination with another antibody known to bind MHC class I antigens to alter the antigens on the cell surface. Suitable methods for altering a surface antigen on a cell for transplantation are described in greater detail in Faustman and Coe (1991) Science 252:1700-1702 and PCT publication WO 92/04033. Methods for altering multiple epitopes on a surface antigen on a cell for transplantation are described in greater detail in PCT publication WO 95/26741, the contents of which are incorporated herein by reference.

The altered (also referred to herein as “modified”) porcine spinal cord cells can comprise a population of cells. The term “population” as used herein refers to a group of two or more cells obtained from the spinal cord. The population of porcine spinal cord cells of the present invention need not contain exclusively cells which are uniform in morphology and function. The population of spinal cord cells can comprise both neural and glial cells. The presence of nonneural cells in the population of spinal cord cells can promote survival and growth of the neural cells upon implantation into a recipient subject. For example, glial cells can provide neurotrophic factors or substrates for spinal cord migration and remyelination.

The modified or unmodified cells described herein can be grown as a cell culture, i.e., as a population of cells which grow in vitro, in a medium suitable to support the growth of the cells. Media which can be used to support the growth of porcine spinal cord cells include mammalian cell culture media, such as those produced by Gibco BRL (Gaithersburg, Md.). See 1994 Gibco BRL Catalogue & Reference Guide. In addition, other substrates upon which the spinal cells can grow including, for example, collagen, collagen plus poly-ornithine and poly-ornithine plus fibronectin, can be used. The medium can be serum-free or supplemented with animal serum such as fetal calf serum. Moreover, growth factors, e.g., neurotrophic factors, can be added to the cell culture to promote spinal cell growth in vitro. Examples of neurotrophic factors include glial cell line-derived growth factor, brain-derived neurotrophic factor, platelet-derived growth factor, neural growth factor, ciliary neurotrophic factor, midkine, insulin-like growth factor I and II, insulin, fibroblast growth factor, neurotrophin-3, neurotrophin 4/5 and transforming growth factor β.

For use in transplantation studies and treatment of spinal cord damage resulting from insult and/or neurodegeneration in humans, the altered porcine spinal cord cells of the present invention are isolated at an appropriate stage of development in order to allow for growth, reproduction, and differentiation following transplantation into a xenogeneic subject. Such spinal cord damage can result from insult and/or neurodegeneration due injury or neurodegenerative disorders. For example, the cells of the invention can be used to reestablish lost or degenerating neural circuitry due to spinal cord injury, amyotrophic lateral sclerosis (ALS), multiple sclerosis (MS), syringomyelia, spinal tumors and metastasis, and spinal infections (e.g., parasitic and bacterial infections) in humans. Preferred porcine spinal cord cells are, therefore, embryonic spinal cord cells, for use in treating spinal cord damage due to insult and/or neurodegeneration. To provide for growth, reproduction, and differentiation of porcine spinal cord cells upon transplantation into a recipient subject, an optimal donor is selected. Typically, spinal cord cells of the invention are porcine embryonic cells which are isolated from porcine fetuses which display the desired characteristics for transplantation. The preferred morphology of spinal cord cells is the characteristic normal morphology of a neuronal cell including a small rounded cell body which does not adhere to the culture vehicle, e.g., culture dish, and glial cells, which tend to have a cell body that is relatively flat. Normal neuron morphology also generally includes the presence of neurite processes. Thus, it is preferred that at least about 1%, more preferably at least about 10%, yet more preferably at least about 20%, still more preferably at least about 30%, and most preferably at least about 40% of the spinal cord cells in culture have the characteristic neuron morphology at the time they are harvested for transplantation. The spinal cord cell cultures of the present invention can also include additional cell types, such as glial cells, as described herein. Preferred spinal cord cells also express distinct immunological markers which distinguish them from other cell types such as connective tissue cells and fibroblasts. For example, the spinal cord cells of the invention do not express fibronectin or keratin which are expressed by connective tissue cells and fibroblasts. In addition, various methods of increasing the survival of the transplanted cells are described herein.

B. Selecting Spinal Cord Cells of the Appropriate Age for Transplantation

The spinal cord consists of several cells types including neural cells (e.g., motor neurons, sensory neurons and GABA-producing intemeurons) and glial cells (e.g., oligodendrocytes and astrocytes). Although various spinal cord cells including nonadrenergic neurons and astrocytes from animals other than swine have been used in transplantation studies (See, e.g., Ribotta (1996) Brain Research 707:245-255 (rat adrenergic neurons); and Wang et al (1995) Neuroscience 65(4):973-981 (rat astrocytes)), the optimal embryonic stage for isolation of porcine spinal cord cells suitable for transplantation into human subjects was determined experimentally. For example, as shown by a comparison of pig and rat fetal development, rat embryonic development follows a different course than pig. However, based on the gestational age for isolating rat cells, one would select a gestational age for isolating pig cells which was significantly older than the optimal age as determined experimentally. The optimal donor age in the rat for spinal cord tissue is between 13 and 15 days of development which corresponds to about 65% of the total gestational time. The equivalent stage in the pig would be about 70 days. However, it was experimentally determined that the optimal age for isolation of spinal cord cells from embryonic pigs is between about twenty (20) and about fifty (50) days, preferably between about twenty (20) and forty (40) days, or between about twenty (20) and about thirty (30) days, or between about twenty-five (25) and about twenty-nine (29) days of gestation. This preferred gestational age for spinal cord cell isolation was determined experimentally by determining the viability of the spinal cord cells after isolation. The results of these studies demonstrate that there is a sharp decline in the viability of isolated spinal cord cells in fetuses older than about thirty (30) to thirty-five (35) days of development. Additionally, cells with viabilities below 50% generally do not give rise to viable grafts. Therefore, embryonic swine older than thirty (30) days are not the preferred source of spinal cord cells for transplantation into humans. In embryonic swine younger than about twenty-four (24) days, the connective tissue is not as easily separated from the desired tissue. Thus, embryonic porcine spinal cord cells suitable for transplantation into humans are preferably obtained from embryonic pigs between about twenty (20) and about fifty (50) days, preferably between about twenty (20) and forty (40) days, or between about twenty (20) and about thirty (30) days, or preferably between about twenty-five (25) and about twenty-nine (29) days of gestation.

In one aspect, the invention includes an isolated porcine spinal cord cell which, upon transplantation into a xenogeneic subject, promotes or enhances neural sparing and/or enhances locomotor function. Another aspect of the invention includes an isolated porcine spinal cord cell which, upon transplantation into a xenogeneic subject, reduces or prevents neurodegeneration of surrounding cells within the host's spinal cord.

C. An Isolated Population of Porcine Spinal Cord Cells

This invention also features an isolated population of cells obtained from porcine spinal cord. For example, spinal cord cells from the mid-lumbar to the thoracic region can be isolated for use in the invention. As used herein, the term “isolated” refers to a cell or population of cells which has been separated from its natural environment. This term includes gross physical separation from its natural environment, e.g., removal from the donor animal, e.g., a pig, and alteration of the cell's relationship with the neighboring cells with which it is in direct contact by, for example, dissociation. The term “isolated” does not refer to a cell or population of cells which is in a tissue section, is cultured as part of a tissue section, or is transplanted in the form of a tissue section. When used to refer to a population of porcine spinal cord cells, the term “isolated” includes populations of cells which result from proliferation of the isolated cells of the invention. The term “population” is intended to include a group of two or more cells. Cells in a population of cells can be obtained from the same or different source(s), e.g., the same swine or several different swine. However, the cells are not necessarily of the same cell type. Cells obtained from porcine spinal cord can include, for example, neural cells, neural progenitor cells, glial cells and glial progenitor cells. Progenitor or precursor cells can be distinguished from committed cells by, for example, differential staining. For example, neural and glial cell precursors express vimentin and can be identified with a vimentin-specific stain while mature neural cells and glial cells do not generally express vimentin. Thus, in one embodiment, the isolated population of spinal cord cells obtained from embryonic porcine spinal cord includes spinal cord progenitor cells. The embryonic porcine spinal cord cells are preferably obtained from embryonic pigs between about twenty (20) and about fifty (50) days, preferably between about twenty (20) and forty (40) days, or between about twenty (20) and about thirty (30) days, or between about twenty-five (25) and about twenty-nine (29) days.

In addition, the various spinal cord cells can be distinguished from each other by staining for cell-type specific markers. For example, motor neurons express choline acetyltransferase, sensory neurons express serotonin receptors, GABAergic spinal cord interneurons express glutamic acid decarboxylase, and oligodendrocytes express galactocerebrosidase.

D. Spinal Cord Cells Isolated from an Essentially Pathogen-Free Swine

In another embodiment, the spinal cord cells of the invention are cells determined to be free from at least one organism which originates in the animal from which the cells are obtained and which transmits infection or disease to a recipient subject. Spinal cord cells with these characteristics can be obtained by screening the animal to determine if it is essentially free from organisms or substances which are capable of transmitting infection or disease to a xenogeneic recipient, e.g., a human recipient, of the cells. Typically, the cells are porcine cells which are obtained from a swine which predetermined to be essentially free from pathogens which detrimentally affect humans. For example, the pathogens from which the swine are free include, but are not limited to, one or more of pathogens from the following categories of pathogens: zoonotic, cross-placental and neurotropic organisms. As used herein, “zoonotic” refers to organisms which can be transferred from pigs to man under natural conditions; “cross-placental” refers to organisms capable of crossing the placenta from mother to fetus; and “neurotropic” refers to organisms which selectively infect neural cells. Within each of these categories, the organism can be a parasite, bacterium, mycoplasma, and/or virus. For example, the swine can be free from parasites such as zoonotic parasites (e.g., toxoplasma), cross-placental parasites (e.g., eperythozoon suis or toxoplasma), neurotropic parasites (e.g., toxoplasma), and/or mycoplasma, such as M. Hypopneumonia. Additionally, the swine can be free from bacteria such as zoonotic bacteria (e.g., brucella, listeria, mycobacterium TB, leptospirillum), cross-placental bacteria (e.g., brucella, listeria, leptospirillum) and/or neurotropic bacteria (e.g., listeria). Specific examples of bacteria from which the swine can be free include brucella, clostridium, hemophilus suis, listeria, mycobacterium TB, leptospirillum, salmonella and hemophilus suis. Additionally, the swine can be free from viruses such as zoonotic viruses, viruses that can cross the placenta in pregnant sows and neurotropic viruses. Zoonotic viruses include, for example, a virus in the rabies virus group, a herpes-like virus which causes pseudorabies, encephalomyocarditis virus, swine influenza Type A, transmissible gastroenteritis virus, parainfluenza virus 3 and vesicular stomatitis virus. Cross-placental viruses include, for example, viruses that cause porcine respiratory reproductive syndrome, a virus in the rabies virus group, a herpes-like virus which causes pseudorabies, parvovirus, a virus that causes swine vesicular disease, teschen (porcine polio virus), hemmaglutinating encephalomyocarditis, cytomegalovirus, suipoxvirus, and swine influenza type A. Neurotropic viruses include, for example, a virus in the rabies virus group, a herpes-like virus which causes pseudorabies, parvovirus, encephalomyocarditis virus, a virus which causes swine vesicular disease, porcine poliovirus (teschen), a virus which causes hemmaglutinating encephalomyocarditis, adenovirus, parainfluenza 3 virus. Specific examples of viruses from which the swine are free include: a virus which causes (or results in) porcine respiratory reproductive syndrome, a virus in the rabies virus group, a herpes-like virus which causes pseudorabies, parvovirus, encephalomyocarditis virus, a virus which causes swine vesicular disease, porcine poliovirus (teschen), a virus which causes hemmaglutinating encephalomyocarditis, cytomegalovirus, suipoxvirus, swine influenza type A, adenovirus, transmissible gastroenteritis virus, a virus which causes bovine viral diarrhea, parainfluenza virus 3, and vesicular stomatitis virus.

In one embodiment, the pigs from which the spinal cord cells are isolated are essentially free from the following organisms: Toxoplasma, eperythrozoon, brucella, listeria, mycobacterium TB, leptospirillum, hemophilus suis, M. Hypopneumonia, a virus which causes porcine respiratory reproductive syndrome, a virus which causes rabies, a virus which causes pseudorabies, parvovirus, encephalomyocarditis virus, a virus which causes swine vesicular disease, porcine polio virus (teschen), a virus which causes hemagglutinating encephalomyocarditis, suipoxvirus, swine influenza type A, adenovirus, transmissible gastroenteritis virus, a virus which causes bovine viral diarrhea, and vesicular stomatitis virus. The phrase “essentially free from organisms or substances which are capable of transmitting infection or disease to a xenogeneic recipient” (also referred to herein as “essentially pathogen-free”) when referring to a swine from which cells are isolated or to the cells themselves means that swine does not contain organisms or substances in an amount which transmits infection or disease to a xenogeneic recipient, e.g. a human. Example III provides representative, but not limiting, examples of methods for selecting swine which are essentially free from various organisms. The cells of the invention can be isolated from embryonic or post-natal swine which are determined to be essentially free of such organisms. These swine are maintained under suitable conditions until used as a source of cells for transplantation.

Optimal gestational ages of the swine from which these cells are isolated are described in detail herein. Porcine spinal cord cells isolated from essentially pathogen-free swine can additionally be modified to reduce the immunogenecity of the cells upon transplantation into a xenogeneic subject as described herein.

### II. Methods of the Invention

A. Method for Isolating Porcine Spinal Cord Cells from an Essentially Pathogen-Free Swine

Another aspect of the invention pertains to a method for isolating a spinal cord cell from a pig which is essentially free from organisms or substances which are capable of transmitting infection or disease to a xenogeneic recipient of the cells. According to the method, swine are tested for the presence or absence of organisms or substances which are capable of transmitting infection or disease to a xenogeneic recipient, e.g., a human recipient, of the cells. Such organisms include, but are not limited to, one or more of pathogens from the following categories of pathogens: zoonotic, cross-placental and neurotropic organisms. Within each of these catagories, the organisms can be parasites, bacteria, mycoplasma, and/or viruses. The swine can be free from, for example, parasites such as toxoplasma and eperytherozoon, or mycoplasma, such as M. Hypopneumonia. Examples of bacteria from which the swine can be free include brucella, listeria, mycobacterium TB, leptospirillum, and hemophilus suis. Additionally, the swine can be free from viruses such as zoonotic, cross placenta, and neurotrophic viruses. Specific examples of viruses from which the swine are free include: a virus which causes (or results in) porcine respiratory reproductive syndrome, a virus in the rabies virus group, a herpes-like virus which causes pseudorabies, parvovirus, encephalomyocarditis virus, a virus which causes swine vesicular disease, porcine poliovirus (teschen), a virus which causes hemmaglutinating encephalomyocarditis, cytomegalovirus, suipoxvirus, swine influenza type A, adenovirus, transmissible gastroenteritus virus, a virus which causes bovine viral diarrhea, parainfluenza virus 3, and vesicular stomatitis virus. Preferably, spinal cord cells are isolated from embryos of swine which are essentially free of these organisms.

Methods for isolating spinal cord cell tissue are known in the art. For example, the spinal cord can be isolated from surrounding body tissue and then solid spinal cord tissue samples can be dissected from surrounding meninges, e.g., by dissecting the tissue under a microscope. The cells in the spinal cord tissue sample can then be dissociated by mechanical means, e.g., chopping and/or successive pipette trituration, or by chemical means, e.g., enzymes. The swine which are employed in the method of the invention as a source of spinal cord cells include embryonic swine (swine fetuses) and postnatal swine. If an embryonic swine is to be used as a source of spinal cord cells, semen from a boar which has been tested to be essentially free of disease transmitting organisms is employed to artificially inseminate a female swine which is essentially free from such organisms. At a selected gestational age, described herein, a hysterectomy is performed under appropriate conditions of sterility and the fetuses are thereafter removed in their individual amniotic sacs. Appropriate spinal cord cells or tissue are thereafter recovered under appropriate conditions of sterility.

The swine which are essentially free from organisms or substances which transmit infection or disease to a recipient subject can be employed as a source of a variety of spinal cord cells. Porcine spinal cord cells isolated from essentially pathogen-free swine can additionally be modified as described herein.

B. Method for Treating Spinal Cord Damage Due to Insult and/or Neurodegeneration in the Spinal Cord of a Xenogeneic Subject Using Porcine Spinal Cord Cells

A still further aspect of the invention pertains to methods for treating spinal cord damage due to insult and/or neurodegeneration in the spinal cord of a xenogeneic subject, particularly a human subject, in which porcine spinal cord cells are introduced into a damaged area in the spinal cord of the subject. As used herein, the phrase “treat” or “treatment” refers to transplantation of porcine spinal cord cells of the invention into the spinal cord of a human subject having spinal cord damage to thereby treat the spinal cord damage. Preferably, porcine spinal cord cells are introduced into a subject with spinal cord damage in an amount sufficient to reduce or prevent neurodegeneration associated with degenerative disorders and/or promote or enhance functional recovery associated with spinal cord injury. Preferably, treatment according to the method of the invention results in replacement of lost or damaged spinal cord cells. Spinal cord damage includes an impairment or absence of a normal neurological function of the spinal cord due to, for example, spinal cord injury, neurodegenerative disorders and/or aging. In one embodiment, the porcine spinal cord cells used to treat spinal cord damage have, in unmodified form, at least one antigen on the cell surface which is capable of stimulating an immune response against the cell in a xenogeneic subject. Prior to transplantation, the antigen on the cell surface is altered to inhibit rejection of the cell when introduced into the xenogeneic subject.

As used herein, the terms “introducing”, “implanting”, and “transplanting” are used interchangeably. The porcine spinal cord cells of the invention are introduced into a subject by any appropriate route which results in delivery of the cells to a desired location in the subject. For example, a common method of administration of cells into the spinal cord of a subject is by direct stereotaxic injection of the cells into the area of spinal cord damage as well as sites rostral and caudal to that area. See e.g., Stokes and Reier (1992) Experimental Neurology 116:1-12. Cells can be administered in a physiologically compatible carrier, such as a buffered saline solution. To treat spinal cord damage in humans, the dosage of cells can be determined based on dosing experiments in rats. The dosing of human equivalents from rat experiments can be determined by first calculating the optimal dose of cells per lesion volume. The lesion volume is calculated as the gap volume in the severed spinal cord, or the volume of contused tissue, determined by gross anatomy or histological analysis. The appropriate human dose can then be calculated by multiplying the optimal rat dose by the volume of the human lesion, determined by, for example, magnetic resonance imaging (MRI) or gross anatomical examination. For example, if the optimized rat dose is 1×10₆ cells for lesions which are 2 mm³, and the transected spinal cord of the human patient has a gap of 20 mm³, then a dose of 1×10⁷ cells will be applied to the damaged spinal area in the human patient.

The cells of the invention can be administered directly after spinal cord damage, e.g., spinal cord injury, occurs or the introduction of the cells into the damaged spinal area can be delayed. It has been demonstrated in the art that delaying transplantation into an injured spinal cord for periods of time can enhance long term survival of the cells in vivo. For example, Shibayama et al. (1998) Neuroreport 9:11-14, disclose that a delaying transplantation of spinal cord grafts up to 7 days after the injury occurs results in effective long term survival and integration of spinal cord transplants. See also, Hoovler et al. (1991) Acta. Neuropathology 81:303-31 1. Thus, the spinal cord cells of the invention can be administered to a subject directly after spinal cord damage or the administration of the cells can be delayed for a period of time (e.g., a month after injury).

The cells of the invention can be inserted into a delivery device which facilitates introduction by e.g., injection, of the cells into the subjects. Such delivery devices include tubes, e.g., catheters, for injecting cells and fluids into the body of a recipient subject. In a preferred embodiment, the cells of the invention can be introduced into the subject at a desired location using a micropipette. The porcine spinal cord cells of the invention can be inserted into such a delivery device, e.g., a micropipette or syringe, in the form of a solution. Alternatively, the cells can be embedded in a support matrix when contained in such a delivery device. As used herein, the term “solution”includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by using a pharmaceutically acceptable carrier or diluent and, as required, other ingredients enumerated above, followed by filtered sterilization, and then incorporating porcine spinal cord cells as described herein.

Support matrices in which the porcine spinal cord cells can be incorporated or embedded include matrices which are recipient-compatible and which degrade into products which are not harmful to the recipient. Natural and/or synthetic biodegradable matrices are examples of such matrices. Natural biodegradable matrices include collagen matrices. Synthetic biodegradable matrices include synthetic polymers such as polyanhydrides, polyorthoesters, and polylactic acid. A preferred support matrix for use in the present invention is gelfoam. In addition, the cells of the invention can be administered in a guidance channel (e.g., polyacrylonitrile/polyvinylchloride (PAN/PVC) guidance channels), such as those described in Bunge et al. (1994) J. Neurology 241:536-539, which can serve as a guide for regenerating axons.

The methods of the invention are particularly useful for treating human subjects having spinal cord damage. Such spinal cord damage can be the result of disease, injury, and/or aging. As used herein, spinal cord damage includes morphological and/or functional abnormality of a spinal cord cell or a population of spinal cord cells as a result of, for example, spinal cord injury, neurodegenerative disorders and/or aging. Spinal cord injuries include, but are not limited to, damage to the spinal cord resulting from compression (e.g., chronic compression), contusions (bruising), distraction (stretching), solid core lesions caused by central core syndrome, and injuries which sever or partially sever the spinal cord. In addition, spinal cord damage can result from neurodegenerative disorders which cause degeneration of cells of the spinal cord (e.g., motor neurons). Non-limiting examples of morphological and functional abnormalities which can result from spinal cord injuries and neurodegenerative disorders include physical deterioration and/or death of spinal cord cells, abnormal growth patterns of spinal cord cells, abnormalities in the physical connection between spinal cord cells, under- or over production of a substance or substances, e.g., a neurotransmitter, by spinal cord cells, failure of spinal cord cells to produce a substance or substances normally produced, production of substances, e.g., neurotransmitters, transmission of electrical impulses in abnormal patterns or at abnormal times and/or induction of secondary neuronal injury and atrophy. Degeneration can occur in any area of the spinal cord of a subject and is seen with many disorders including, for example, spinal cord injury, ALS, multiple sclerosis, syringomyelia, spinal tumors or metastasis, and spinal cord infections (e.g., parasitic or bacterial infections).

In one embodiment of the invention, porcine spinal cord cells, preferably obtained from embryonic porcine spinal cord at about days twenty-five (25) to twenty-nine (29) of gestation, are transplanted into the spinal cord of a human subject to treat, for example, spinal cord injury. Injury to the spinal cord can result in insult and considerable degeneration and demyelination of axonal projections which can result in ischemia, edema and hemorrhaging and subsequent inflammatory responses. Porcine spinal cord cells obtained at selected gestational ages can be transplanted into the damaged spinal area (i.e., the epicenter) as well as at sites caudal and rostral from the epicenter. Transplantation of porcine spinal cord cells of the invention into the damaged spinal areas populate areas of lost or damaged spinal cord cells. Treatment of spinal cord injury by the methods of the invention can result in the promotion or enhancement of neuronal sparing, reduction in secondary host injury responses and/or an enhancement of functional recovery (i.e., locomotor recovery).

In another embodiment of the invention, porcine spinal cord cells, preferably obtained from embryonic porcine spinal cord at about days twenty-five (25) to twenty-nine (29) of gestation, are transplanted into the spinal cord of a human subject to treat, for example, a neurodegenerative disorder such as ALS. ALS is a progressive neurodegenerative disorder which can result in the selective loss of motor neurons and ultimately paralysis in a human subject. Porcine spinal cord cells at select gestational ages can be transplanted into a subject, i.e., human subject, to reduce or prevent the progression of the symptoms of ALS.

The term “subject” is intended to include mammals, particularly humans, susceptible to injury-, age- and/or disease-related spinal cord damage. The term “subject” also includes mammals in which an immune response is elicited against allogeneic or xenogeneic cells. Examples of subjects include primates (e.g., humans, and monkeys). A “xenogeneic subject” as used herein is a subject into which cells of another species are transplanted or are to be transplanted. Porcine spinal cord cells are introduced into a subject in an amount suitable to reconstitute the damaged spinal area and inhibit or reduce progression of spinal cord neurodegeneration and/or partially re-establish neural circuitry and locomotor recovery.

Prior to introduction into damaged areas in the spinal cord of a subject, the porcine spinal cord cells can be modified to enhance their neuroregenerative capacity and/or inhibit immunological rejection. The porcine spinal cord cells can, as described in detail above, be rendered suitable for introduction into a xenogeneic subject by alteration of at least one immunogenic cell surface antigen (e.g., an MHC class I antigen). To inhibit rejection of transplanted porcine spinal cord cells and to achieve immunological non-responsiveness in an allogeneic or xenogeneic transplant recipient, the method of the invention can include alteration of immunogenic antigens on the surface of the porcine spinal cord cells prior to introduction into the subject. This step of altering one or more immunogenic antigens on porcine spinal cord cells can be performed alone or in combination with administering to the subject of an agent which inhibits T cell activity in the subject. Alternatively, inhibition of rejection of a porcine spinal cord cell graft can be accomplished by administering to the subject an agent which inhibits T cell activity in the subject in the absence of prior alteration of an immunogenic antigen on the surface of the porcine spinal cord cell. As used herein, an agent which inhibits T cell activity is defined as an agent which results in removal (e.g., sequestration) or destruction of T cells within a subject or inhibits T cell functions within the subject (i.e., T cells may still be present in the subject but are in a non-functional state, such that they are unable to proliferate or elicit or perform effector functions, e.g. cytokine production, cytotoxicity etc.). The term “T cell” encompasses mature peripheral blood T lymphocytes. The agent which inhibits T cell activity may also inhibit the activity or maturation of immature T cells (e.g., thymocytes).

A preferred agent for use in inhibiting T cell activity in a recipient subject is an immunosuppressive drug. The term “immunosuppressive drug or agent” is intended to include pharmaceutical agents which inhibit or interfere with normal immune function. A preferred immunsuppressive drug is cyclosporin A. Other immunosuppressive drugs which can be used include FK506, and RS-61443. In one embodiment, the immunosuppressive drug is administered in conjunction with at least one other therapeutic agent. Additional therapeutic agents which can be administered include steroids (e.g., glucocorticoids such as prednisone, methyl prednisolone and dexamethasone) and chemotherapeutic agents (e.g., azathioprine and cyclosphosphamide). In a preferred embodiment, methylprednisolone is administered to the subject after transplnatation of the cells of the invention such that local inflammatory responses are deterred. In another embodiment, an immunosuppressive drug is administered in conjunction with both a steroid and a chemotherapeutic agent. Suitable immunosuppressive drugs are commercially available (e.g., cyclosporin A is available from Novartis, Corp., East Hanover, N.J.).

An immunsuppressive drug is administered in a formulation which is compatible with the route of administration. Suitable routes of administration include intravenous injection (either as a single infusion, multiple infusions or as an intravenous drip over time), intraperitoneal injection, intramuscular injection and oral administration. For intravenous injection, the drug can be dissolved in a physiologically acceptable carrier or diluent (e.g., a buffered saline solution) which is sterile and allows for syringability. Dispersions of drugs can also be prepared in glycerol, liquid polyethylene glycols, and mixtures thereof and in oils. Convenient routes of administration and carriers for immunsuppressive drugs are known in the art. For example, cyclosporin A can be administered intravenously in a saline solution, or orally, intraperitoneally or intramuscularly in olive oil or other suitable carrier or diluent.

An immunosuppressive drug is administered to a recipient subject at a dosage sufficient to achieve the desired therapeutic effect (e.g., inhibition of rejection of transplanted cells). Dosage ranges for immunosuppressive drugs, and other agents which can be coadministered therewith (e.g., steroids and chemotherapeutic agents), are known in the art (See e.g., Freed et al. New Engl. J. Med. (1992) 327:1549: Spencer et al. (1992) New Engl. J. Med. 327:1541; Widner et al. (1992) New Engl. J. Med. 327:1556; Lindvall et al. (1992) Ann. Neurol. 31:155; and Lindvall et al. (1992) Arch. Neurol. 46:615). A preferred dosage range for immunosuppressive drugs, suitable for treatment of humans, is about 1-30 mg/kg of body weight per day. A preferred dosage range for cyclosporin A is about 1-10 mg/kg of body weight per day, more preferably about 1-5 mg/kg of body weight per day. Dosages can be adjusted to maintain an optimal level of the immunosuppressive drug in the serum of the recipient subject. For example, dosages can be adjusted to maintain a preferred serum level for cyclosporin A in a human subject of about 100-200 ng/ml. It is to be noted that dosage values may vary according to factors such as the disease state, age, sex, and weight of the individual. Dosage regimens may be adjusted over time to provide the optimum therapeutic response according to the individual need and the professional judgment of the person administering or supervising the administration of the compositions, and that the dosage ranges set forth herein are exemplary only and are not intended to limit the scope or practice of the claimed composition.

In one embodiment of the invention, an immunsuppressive drug is administered to a subject transiently for a sufficient time to induce tolerance to the transplanted cells in the subject. Transient administration of an immunosuppressive drug has been found to induce long-term graft-specific tolerance in a graft recipient (See Brunson et al. (1991) Transplantation 52:545; Hutchinson et al. (1981) Transplantation 32:210; Green et al. (1979) Lancet 2:123; Hall et al. (1985) J. Exp. Med. 162:1683). Administration of the drug to the subject can begin prior to transplantation of the cells into the subject. For example, initiation of drug administration can be a few days (e.g., one to three days) before transplantation. Alternatively, drug administration can begin the day of transplantation or a few days (generally not more than three days) after transplantation. Administration of the drug is continued for sufficient time to induce donor cell-specific tolerance in the recipient such that donor cells will continue to be accepted by the recipient when drug administration ceases. For example, the drug can be administered for as short as three days or as long as three months following transplantation. Typically, the drug is administered for at least one week but not more than one month following transplantation. Induction of tolerance to the transplanted cells in a subject is indicated by the continued acceptance of the transplanted cells after administration of the immunosuppressive drug has ceased. Acceptance of transplanted tissue can be determined morphologically (e.g., with skin grafts by examining the transplanted tissue or by biopsy) or by assessment of the functional activity of the graft.

Another type of agent which can be used to inhibit T cell activity in a subject is an antibody, or fragment or derivative thereof, which depletes or sequesters T cells in a recipient. Antibodies which are capable of depleting or sequestering T cells in vivo when administered to a subject are known in the art. Typically, these antibodies bind to an antigen on the surface of a T cell. Polyclonal antisera can be used, for example anti-lymphocyte serum. Alternatively, one or more monoclonal antibodies can be used. Preferred T cell-depleting antibodies include monoclonal antibodies which bind to CD2, CD3, CD4 or CD8 on the surface of T cells. Antibodies which bind to these antigens are known in the art and are commercially available (e.g., from American Type Culture Collection). A preferred monoclonal antibody for binding to CD3 on human T cells is OKT3 (ATCC CRL 8001). The binding of an antibody to surface antigens on a T cell can facilitate sequestration of T cells in a subject and/or destruction of T cells in a subject by endogenous mechanisms. Alternatively, a T cell-depleting antibody which binds to an antigen on a T cell surface can be conjugated to a toxin (e.g., ricin) or other cytotoxic molecule (e.g., a radioactive isotope) to facilitate destruction of T cells upon binding of the antibody to the T cells. See PCT publication WO 95/26740, for further details concerning the generation of antibodies which can be used in the present invention.

Another type of antibody which can be used to inhibit T cell activity in a recipient subject is an antibody which inhibits T cell proliferation. For example, an antibody directed against a T cell growth factor, such as IL-2, or a T cell growth factor receptor, such as the IL-2 receptor, can inhibit proliferation of T cells (See e.g., DeSilva, D. R. et al. (1991) J. Immunol. 147:3261-3267). Accordingly, an IL-2 or an IL-2 receptor antibody can be administered to a recipient to inhibit rejection of a transplanted cell (see e.g. Wood et al. (1992) Neuroscience 49:410). Additionally, both an IL-2 and an IL-2 receptor antibody can be coadministered to inhibit T cell activity or can be administered with another antibody (e.g., which binds to a surface antigen on T cells).

An antibody which depletes, sequesters or inhibits T cells within a recipient can be administered at a dose and for an appropriate time to inhibit rejection of cells upon transplantation. Antibodies are preferably administered intravenously in a pharmaceutically acceptable carrier or diluent (e.g., a sterile saline solution). Antibody administration can begin prior to transplantation (e.g., one to five days prior to transplantation) and can continue on a daily basis after transplantation to achieve the desired effect (e.g., up to fourteen days after transplantation). A preferred dosage range for administration of an antibody to a human subject is about 0.1-0.3 mg/kg of body weight per day. Alternatively, a single high dose of antibody (e.g., a bolus at a dosage of about 10 mg/kg of body weight) can be administered to a human subject on the day of transplantation. The effectiveness of antibody treatment in depleting T cells from the peripheral blood can be determined by comparing T cell counts in blood samples taken from the subject before and after antibody treatment. Dosage regimes may be adjusted over time to provide the optimum therapeutic response according to the individual need and the professional judgment of the person administering or supervising the administration of the compositions. Dosage ranges set forth herein are exemplary only and are not intended to limit the scope or practice of the claimed composition.

In another embodiment, the porcine spinal cord cells (e.g., neural and glial cells) of the invention are genetically engineered to express and/or secrete a foreign molecule (e.g., a neurotrophic factor, a neurotransmitter, or a neuroprotective agent), e.g., to enhance their neuroregenerative capacity. In addition, unmodified or modified porcine spinal cord cells can be introduced into the spinal cord of a xenogeneic subject together with other types of cells (e.g., other cells of the nervous system or cells derived from other sources) which have been genetically modified to perform a useful function. For example, in order to promote growth of neurons in a damaged area of the spinal cord of a subject, the spinal cord cells derived from the porcine spinal cord can be implanted into the damaged spinal area together with other cells which have been modified to secrete, for example, a neurotrophic factor. Examples of cells that act as carriers of transgenes to the brain of a subject include fibroblasts (Fisher, L. J. et al. (1991) Neuron 6:371-380; Rosenberg, M. B. et al. (1988) Science 242:1575-1578), adrenal chromaffin cells (Cunningham, L. A. et al. (1991) Brain Res. 561:192-202), astrocytes (Suhr, S. T. and Gage, F. H. (1993) Arch. Neurol. 50(11):1252-1268), and myoblasts (Jiao, S. et al. (1993) Nature 362:450-453; Jiao, S. et al. (1992) Brain Res. 575:143-147; Jiao, S. et al. (1992) Hum. Gene Ther. 3:21-33). Such cells, e.g., fibroblasts and glial cells, can also be used to deliver retroviruses containing genes, e.g., herpes simplex thymidine kinase gene, the gene products of which are targets for other therapeutic drugs or agents, e.g., ganciclovir, to target cells, e.g., tumor cells, to inhibit their growth. Culver, K. et al. (1992) Science 256:1550-1552; Chen, S-H. et al. (1994) Proc. Natl. Acad. Sci. USA 91:3054-3057. Alternatively, the spinal cord cells derived from porcine spinal cord which are to be implanted into a damaged spinal area can themselves be genetically modified to produce, for example, a neurotrophic factor to enhance the growth and development of the implant.

There are several mechanisms by which insult and/or neurodegeneration can be treated using the methods of the present invention or in conjunction with the methods of the present invention. For example, a new function can be introduced into a target cell (e.g., a damaged spinal cord cell) in a phenotypically useful way. A new function can be expressed in such defective target cells (e.g., damaged spinal cord cells) by introducing a genetically modified cell (e.g., porcine spinal cord cells, fibroblasts, myoblasts, etc.) that can establish a tight junction or other contacts with the target cell. Some such contacts are known to permit the efficient diffusion of metabolically important small molecules from one cell to another, leading to phenotypic changes in the recipient cell. Loewenstein, W. R. (1979) Biochim. Biophys. Acta. 560:1-66. This process has been called “metabolic co-operation” and is known to occur between fibroblasts and glial cells. Gruber, H. E. et al. (1985) Proc. Natl. Acad. Sci. USA 82:6662-6666. This type of co-operativity has been demonstrated with CNS cells, as in the case of NGF-mediated protection of cholinergic spinal cord death following CNS damage. Hefti, F. (1986) J. Neurosci. 6:2155; Williams, L. R. et al. (1986) Proc. Natl. Acad. Sci. USA 83:9231-9235.

Another mechanism by which insult and/or degeneration of the spinal cord can be treated using the methods of the invention includes the generation of still other genetically modified cells which can express and secrete a diffusible gene product that can be taken up and used by nearby target cells. One strategy that has been pursued in animal models of neurodegenerative disease is to augment neurotransmitter function within the brain through tissue transplantation. For example, fibroblast cell lines have been modified to express choline acetyltransferase. The modified fibroblasts have then been implanted into the hippocampus of rats where they continue to produce and release acetylcholine after grafting. Fisher, L. J. et al. (1993) Ann. N. Acad. Sci. 695:278-284. Fibroblasts have also been genetically modified to produce tyrosine hydroxylase (an enzyme that converts tyrosine to L-DOPA) and implanted into the striatum of recipient rats with a prior 6-hydroxydopamine lesion. The implanted fibroblasts continue to convert tyrosine to L-DOPA in the host striatum and to affect the host brain as assessed through behavioral measurements. Fisher, L. J. et al. (1991) Neuron 6:371-380.

Another strategy that has been pursued in animal models of spinal cord damage is to deliver neurotrophic factors, such as nerve growth factor (NGF), which sustains the growth and development of neurons, prevents damage-induced death, and attracts the growth of developing or regenerating axons, to the damaged spinal area. Fibroblasts can be modified to secrete NGF. When these fibroblasts are introduced into striatum of a subject such as a rat, they protect neurons from excitotoxin-induced lesions. Schumacher, J. M. et al. (1991) Neuroscience 45(3):561-570.

A cell to be introduced into the subject can be genetically modified in vitro prior to transplantation, or alternatively, the cell can be directly modified in vivo following transplantation. Suhr, S. T. and Gage, F. H. (1993) Arch. Neurol. 50(11):1252-1268; Gage, F. H. et al. (1987) Neuroscience 23(3):795-807. Various methods are available for genetically modifying donor cells such as porcine spinal cord cells, prior to implantation into a recipient subject. These methods include direct DNA uptake (transfection), and infection with viral vectors such as retrovirus, herpes virus, adenovirus, and adeno-associated virus vectors. Suhr, S. T. et al. (1993) Arch. Neurol. 50:1252-1268. Transfection can be effected by endocytosis of precipitated DNA, fusion of liposomes containing DNA or electroporation. Suhr, S. T. et al. (1993) Arch. Neurol. 50:1252-1268. Another method of transfecting donor cells is through the use of a “gene gun”. In this method, microscopic DNA-coated particles are accelerated at high speeds through a focusing tube and “shot” or injected into cells in vitro (Klein, R. M. et al. (1992) Biotechnology 24:384-386; Zelenin, A. V. et al. (1989) FEBS Lett. 244:65-67) or in vivo (Zelenin, A. V. et al. (1991) FEBS Lett. 280:94-96). The cells close around the wound site and express genes carried into the cell on the particles.

Retroviral vectors typically offer the most efficient and best characterized means of introducing and expressing foreign genes in cells, particularly mammalian cells. These vectors have very broad host and cell type ranges, integrate by reasonably well understood mechanisms into random sites in the host genome, express genes stably and efficiently, and under most conditions do not kill or obviously damage their host cells. The methods of preparation of retroviral vectors have been reviewed extensively in the literature (Suhr, S. T. and Gage, F. H. (1993) Arch. Neurol. 50(11):1252-1258; Ray, J. and Gage, F. H. (1992) Biotechniques 13(4):598-603; Anderson, W. F. (1984) Science 226:401-409; Constantini, F. et al. (1986) Science 233:1192-1194; Gilboa, E. et al. (1986) Biotechniques 4:504-512; Mann, R. et al. (1983) Cell 33:153-159; Miller, A. D. et al. (1985) Mol. Cell Biol. 5:431-437; and Readhead, C. et al. (1987) Cell 48:703-712) and are now in common use in many laboratories. Other techniques for producing genetically modified cells are described in detail in PCT publication WO 95/27042 and U.S. Pat. No. 5,082,670, the contents of which are incorporated herein by reference.

C. Methodfor Treating Spinal Cord Damage in the Spinal Cord of a Xenogeneic Subject Using Porcine Spinal Cord Cells

The spinal cord cells of the invention can be isolated by dissecting out the spine of a fetal pig using a dissecting microscope. The mid-lumbar to thoracic region can be mechanically sectioned and the meninges can be teased away from the underlying spinal cord. Once the spinal cord is isolated, the spinal cord cells are dissociated under conditions suitable for isolation of fetal porcine spinal cord cells. Under these conditions, the ingredients of the solution in which the cells are dissociated are adjusted to maintain the highest percentage of viable spinal cord cells. For example, in a preferred dissociation solution, Hank's balanced salt solution without calcium, magnesium, bicarbonate and phenol red is used, as these ingredients have been found to reduce the percentage of viable cells after dissociation. In addition, precautions are taken to reduce the amount of shear strain placed on the cells during dissociation. These precautions include minimizing the generation of air bubbles during dissociation and gently triturating the cells through pipettes with gradually decreasing pipette bore sizes.

Modified or unmodified porcine spinal cord cells, and in particular embryonic porcine spinal cord cells, can used to treat spinal cord damage resulting from insult and/or neurodegeneration, such as that which occurs in human subjects with ALS. Models of spinal cord damage in several different animals have been developed in which the porcine spinal cord cells of the invention can be transplanted to assess their neuroregenerative capacity. For example, mouse models of ALS have been described in which effective therapies are predictive of therapeutic efficacy in humans. In addition, rat models of hemisected and contused spinal cord injuries have been developed which are predictive of these types of spinal cord injury in humans. See e.g., Bregman et al. (1993) Exp. Neurol. 123:3-16 (hemisected rat model), and Basso et al. (1996) Exp. Neurol. 139:24-240-256 (NYU-weight drop contusion impacter model).

As an illustrative example, Gurney et al. have generated a model of ALS disease in a transgenic mouse which expresses a human Cu/Zn superoxide dismutase mutation (SOD mice). Gurney et al. (1994) Science 264:1772-1775. The SOD transgenic mouse model displays a neuropathology resembling the pathogenesis of ALS in humans. To assess therapeutic strategies, porcine spinal cord cells, and in particular embryonic porcine spinal cord cells can be introduced into the damaged spinal area of SOD mice. Morphological and immunohistochemical studies can then be performed by conventional techniques to determine whether the porcine spinal cord implant has integrated, both morphologically and functionally, into the surrounding tissue. Moreover, the ability of the transplant to prevent the death of this mouse model serves as an indication that the progression of neurodegeneration as been inhibited. Example III in the present application describes transplantation of spinal cord cells of the invention into the spinal cord of SOD mice which resulted in the survival of the transplant recipients.

D. Methodfor Treating Spinal Cord Damage Due to Insult and/or Neurodegeneration in the Spinal Cord of a Xenogeneic Subject Using Porcine Spinal Cord Cells Obtained from an Essentially Pathogen-Free Swine

Another method disclosed herein for treating spinal cord damage due to insult and/or neurodegeneration in the spinal cord of a xenogeneic subject includes introducing spinal cord cells obtained from a pig which is essentially free from organisms or substances which are capable of transmitting infection or disease to the subject into a damaged spinal area in the spinal cord of a xenogeneic subject. Swine which are essentially free from organisms or substances which are capable of transmitting infection or disease to a recipient subject are described above under the headings “A Porcine Spinal Cord Cell Isolated from an Essentially Pathogen-Free Swine” and “Method for Isolating a Porcine Spinal Cord Cell from an Essentially Pathogen-Free Swine”. Spinal Cord Damage and neurodegeneration resulting, for example, from spinal cord injury or neurodegenerative disorders are described above under the heading “Method for Treating Spinal Cord Damage in the Spinal Cord of a Xenogeneic Subject Using Modified Porcine Spinal Cord Cells”.

E. Methods for Increasing Survival of Porcine Spinal Cord Cells Introduced into Areas of Insult and/or Neurodegeneration in the Spinal Cord of a Subject

The porcine cells of the present invention can be incubated and/or treated at any stage in their preparation for transplantation, e.g., during dissection, trypsinization, dissociation and plating, and/or production of cell suspensions for transplantation, with a number of agents or factors which promote the survival, growth and differentiation of the cells in vitro and/or in vivo, i.e., in the recipient subject. In one embodiment, such agents or factors can be added at the site of transplantation in the recipient subject after the cells of the invention have been transplanted therein. In some instances, these agents can, for example, minimize or counteract detrimental effects on the cells resulting from the procedures used to prepare the cells for transplantation. For example, porcine cells, when isolated from the donor pigs and prepared for transplantation, may experience cellular trauma and/or hypoxia which lead to the production of reactive oxygen species (ROS) such as superoxide radical anion, hydrogen peroxide, and the hydroxyl free radical. Colton, C. A. et al. (1995) Exp. Neurol. 132:54-61. ROS are known to adversely affect neural function, most likely by affecting a variety of membrane and intracellular components including ion channels, membrane lipids, transport mechanisms such as the NA/K ATPase and Na+/glutamate exchange transport and cytosolic enzymes such as glutamine synthase. Colton, C. A. et al. (1995) Exp. Neurol. 132:54-61. Acute exposure of nerve terminals to ROS results in failure of neurotransmission. Colton, C. A. et al. (1991) Free Rad. Res. Commun. 14:385-393; Colton, C.A. et al. (1989) Free Rad. Biol. Med. 7:3-8. Long term exposure of nerve terminals to ROS results in retraction of neurites and eventually, neuronal death. Halliwell, B. et al. Free Radicals in Biology and Medicine, 2nd ed. (Clarendon Press, Oxford, England 1989). In addition, it is known that ROS provoke membrane lipid peroxidation, consequently reducing the survival of spinal cord cells, i.e., neural cells, in the transplants.

To minimize and/or counteract the adverse effects of these types of oxidative stress during preparation for transplantation, the cells of the present invention can be incubated and/or treated with antioxidants at any stage during the preparation. Examples of such antioxidants include the enzyme antioxidants superoxide dismutase (SOD) and glutathione peroxidase (Colton, C. A. et al. (1995) Exp. Neurol. 132:54-61) which are commercially available from Boehringer Mannheim (Indianapolis, Ind.) and Sigma Chemical Company (St. Louis, Mich.), respectively, agents which promote glutathione formation, e.g. N-acetyl cysteine (NAC), also commercially available from Sigma, and other known antioxidants such as lazaroids, e.g., U-74389G and U-83836E, which are available from Upjohn (Nakao, N. et al. (1994) Proc. Natl. Acad. Sci. USA 91:12408-12412; Frodl, E.M. et al. (1994) NeuroReport 5:2393-2396). Antioxidant enzymes, such as SOD, scavenge ROS and prevent the reaction of superoxide with nitric oxide to form peroxynitrite anion, which has been shown to be toxic to cultured neurons. Nakao, N. et al. (1995) Nature Medicine 1(3):226-23 1. These enzymes can be incubated with the cells of the invention as described above. Another method of introducing these enzymes into the cellular preparations of the present invention is to genetically modify the cells to contain the nucleic acid encoding such enzymes. The genetically modified cells can then produce agents which enhance the survival, growth, and differentiation of the grafted cells in the recipient subject. For example, porcine cells of the invention can be transfected with the human gene for Cu/Zn superoxide dismutase, a pivotal enzyme in the detoxification of oxygen free radicals, (Nakao, N. et al (1995) Natue Medicine 1(3):226-23 1). These transfected cells then express SOD and, consequently, efficiently detoxify ROS generated during tissue preparation and implantation to thereby increase graft survival.

Lazaroids are 21-aminosteroids that lack glucocorticoid activity and are specifically designed to localize within cell membranes and inhibit lipid peroxidation (stabilize membranes by inserting their lipophilic portion into the phospholipid bilayer (Nakao, N. et al. (1994) Proc. Natl. Acad. Sci. USA 91:12408-12412; Frodl, E. M. et al. (1994) NeuroReport 5:2393-2396). Lazaroids are also known to scavenge free radicals, in particular, hydroxyl radicals. Other examples of antioxidants which can be added to the cell cultures and cell suspensions include TGFβ (Prehn, J. H. M et al. (1994) Proc. Natl. Acad. Sci. USA 91:12599-12603), vitamin E (Nakao, N. et al. (1995) Natue Medicine 1(3):226-231), vitamin C, beta carotene, and other compounds which scavenge ROS, inhibit the production of ROS, and/or inhibit lipid peroxidation.

In addition, the oxidative environment of the cells in vitro can be modified to inhibit cellular oxidative stress. For example, during preparation of the porcine cells for transplantation, the partial pressure of oxygen in the cells' environment can be decreased from the normal oxygen partial pressure, i.e., approximately 150 torr O₂, to a decreased oxygen partial pressure, i.e., 38 torr O₂ (about 5% O₂). This method of decreasing oxidative stress can be combined with treatment of the cells with one or more of the above-described antioxidants. For example, the combination of the partial oxygen pressure of 38 torr (e.g., 5% O₂) and treatment with NAC is effective for promoting survival of TH+neurons. Colton, C. A. et al. (1995) Exp. Neurol. 132:54-61.

During the hypoxic conditions associated with the preparation of the cells of the invention for transplantation, the release of excitatory amino acids in the extracellular space stimulates N-methyl-D-aspartate (NMDA) receptors to increase the activity of nitric oxide synthase (NOS) which in turn results in increased biosynthesis of nitric oxide (NO). Nitric oxide is a neurotransmitter which can be toxic under conditions of excessive formation. Dawson, T. et al. (1995) The Neuroscientist 1(1):7-17. The toxic effects of NO occur through an interaction with the superoxide anion to form peroxynitrite, a highly reactive molecule which is able to nitrosylate proteins as well as initiate lipid peroxidation. Peroxynitrite also spontaneously decomposes to the hydroxyl and NO₂ free radicals, which mediate a variety of toxic effects. Dawson, T. et al. (1995) The Neuroscientist 1(1):7-17. Inhibitors of NOS, such as gangliosides, FK506, and cyclosporine A (Dawson, T. et al. (1995) The Neuroscientist 1(1):7-17), can be added to the cell preparations to inhibit the production of NO, thereby decreasing the production of peroxynitrite and its derivatives. Superoxide dismutase is another agent which can decrease the adverse effects of overproduction of NO and the toxic effects it mediates. Dawson, T. et al. (1995) The Neuroscientist 1(1):7-17.

Trauma and its associated adverse effects, e.g., membrane peroxidation, free radical induced cell damage (Gonzalez-Garcia, M. et al. (1995) Proc. Nati. Acad. Sci. USA 92:4304-4308; Zhong, L-T. et al. (1993) Proc. Natl. Acad Sci. USA 90:4533-4537), induced by preparation of the cells of the invention for implantation can also result in programmed cell death (apoptosis) of the transplanted cells. To reduce the occurrence of apoptosis in the transplanted cells, the porcine cells of the invention can be transfected with nucleic acids encoding antiapoptotic gene products such as the bcl-2 (Talley, A. K. et al. (1995) Mol. Cell Biol. 15(5):2359-2366; Merry, D. E. et al. (1994) Development 120:301-311; Prehn, J. H. et al. (1994) Proc. Natl. Acad. Sci. USA 91:12599-12603; Zhong, L-T. et al. (1993) Proc. Natl. Acad. Sci. USA 90:4533-4537), bcl-xL, the bcl-xβ (González-Garcia, M. et al. (1995) Proc. Natl. Acad. Sci. USA 92:4304-4308), and/or the crmA (Talley, A. K. et al. (1995) Mol. Cell Biol. 15(5):2359-2366) gene product. These gene products have been shown to inhibit programmed neural cell death. In addition, the transfected porcine cells of the invention can be treated with agents which upregulate the expression or function of these gene products, e.g., TGFβ1 and TGFβ3 which upregulate the expression of bcl-2 (González-Garcia, M. et al. (1995) Proc. Natl. Acad. Sci. USA 92:4304-4308; Prehn, J. H. et al. (1994) Proc. Natl. Acad. Sci. USA 91:12599-12603) to augment the neuroprotective effect of the antiapoptotic gene products produced by the cells. Other factors, such as nerve growth factor (NGF) and platelet-derived growth factor (PDGF) have been found to have antiapoptotic activity (Zhong, L-T. et al. (1993) Proc. Natl. Acad. Sci. USA 90:4533-4537). The cells of the invention, therefore, can also be transfected with nucleic acid encoding these factors. Enzyme antioxidants, such as superoxide dismutase and catalase (Bonfoco, E. et al. (1995) Proc. Natl. Acad. Sci. USA 92:7162-7166), and other antioxidants, such as NAC (Talley, A. K. et al. (1995) Mol. Cell Biol. 15(5):2359-2366) can also be used to prevent cells of the invention from undergoing programmed cell death during preparation for transplantation.

Other factors, such as neurotrophic factors, which contribute to spinal cord neural cell development, nerve fiber formation, and maintenance of neurons can be added to the cells of the invention in vitro during preparation for transplantation and/or to the cell suspension itself for introduction into the recipient subject along with the cells of the invention. The cells of the invention can also be genetically modified to produce such neurotrophic factors as described herein. The neurotrophic factor which is added to the cells of the present invention can be selected based on the presence of its receptors on the cells which are to be transplanted. Several neurotrophic factors have been found to exert neurotrophic influence on mature central nervous system in vivo, to thereby increase axonal growth into spinal cord transplants. Examples of neurotrophic factors which can be used in the present invention include, for example, brain-derived neurotrophic factor, platelet-derived growth factor, neural growth factor, ciliary neurotrophic factor, midkine, insulin-like growth factor I and II, fibroblast growth factor, neurotrophin-3 and neurotrophin 4/5. See Bregman et al. (1998) Exp. Neurology 149:13-27, and Novikova et al. (1997) Eur. J Neuroscience 9:2774-2777. In addition, it is contemplated that various combinations of neurotrophic factors described herein can act synergistically and, therefore, can be used together to promote survival of the transplanted cells of the invention.

Certain drugs also possess neurotrophic activity. Examples of such drugs include FK506 and cyclosporin A (Lyons, W. E. et al. (1994) Proc. Natl. Acad. Sci. USA 91:3191-3195) which block the neurotoxicity elicited by glutamate acting at N-methyl-D-aspartate (NMDA) receptors by, for example, augmenting phosphorylated levels of NOS. As phosphorylated NOS inhibits its catalytic activity, these drugs effectively reduce NO formation and prevent the neurotoxic effects of NMDA on these cells. Other drugs which possess neurotrophic activity and can be used in the present invention are those small molecules which bind to the same binding proteins as FK506 and/or cyclosporin A and, therefore, mediate similar neuroprotective effects. Lyons, W. E. et al. (1994) Proc. Natl Acad. Sci. USA 91:3191-3195.

It is specifically contemplated herein that combinations of one or more of the above-described agents and factors can be used to promote survival of the cells of the invention prior to or after the cells are transplanted into recipient subjects. For example, cells of the present invention can be contacted with one or more of the agents or factors described herein to promote survival of the cells in vitro and/or in vivo. In another embodiment, the cells of the invention can be transfected with the nucleic acid of one or more of the agents or factors described herein and also contacted with one or more of the agents or factors described herein. Moreover, although many of the neurotrophic factors described herein are specific for a particular cell type, the association of these factors with such a cell type does not exclude the use of that factor with a different cell type. Treatment of the cells of the invention with the agents or factors described herein can occur simultaneously or sequentially.

The present invention is further illustrated by the following examples which in no way should be construed as being further limiting. The contents of all cited references (including literature references, issued patents, published patent applications, and co-pending patent applications) cited throughout this application are incorporated herein by reference.

### EXAMPLES

### Example I

### Isolation of Porcine Spinal Cord Cells

Fetal spinal cord cells were isolated under a dissecting microscope by first dissecting away the spine from the body. The mid-lumbar to thoracic region was cut in two to three sections and each section was dissected independently. The meninges were teased away from the underlying cord and fetal spinal cord tissue from the fetuses were minced and pooled. The fetal tissue was then incubated with 0.5% Trypsin-EDTA in Hanks balanced solution (HBSS). To facilitate dissociation, the tissue was washed in the presence of DNase (Final concentration 50 μg/ml). Following the washing, the tissue was titurated in the presence of DNase by passing it through pulled Pasteur pipettes with decreasing bore size until a single cell suspension is attained. The cells were then washed and re-suspended in the appropriate media for transplantation at a concentration of approximately 100,000 cells per microliter.

### Example II

### Transplantation of Porcine Spinal Cord Cells into Hemisected Rat Model for Spinal Cord Injury

Preparation of Hemisected Rat Models of Spinal Cord Injury

Female Sprague-Dawley rats (250-300 g) were anesthetized with sodium pentobarbital (50 mg/kg) during the surgical procedures. Hemisection was performed on the right half of the spinal cord between thoracic and lumbar regions T13 and L1. Palpation on the dorsal surface mid-section was used to determine the position of the most caudal rib. The last rib served as a guide for the location of the lumbar enlargement. The spinal cord was exposed via laminectomy of several spinal segments (T12-L2). The exposure of these anatomical landmarks assured appropriate locality for ligation. The hemisection was performed with a number 15 scalpel blade and a 1 mm³ piece of the right spinal cord was made. To ensure separation of the transection from the adjacent rostral spinal cord segment, a 28 gauge needle was inserted medially and dragged out laterally through the separated region. Bleeding in the spinal hemisected area was controlled by applying gelfoam (Upjohn, Kalamazoo, Mich.) soaked in bovine thrombin to surrounding bone and soft tissue.

Transplantation of Porcine Spinal Cord Cells into Hemisected Rats

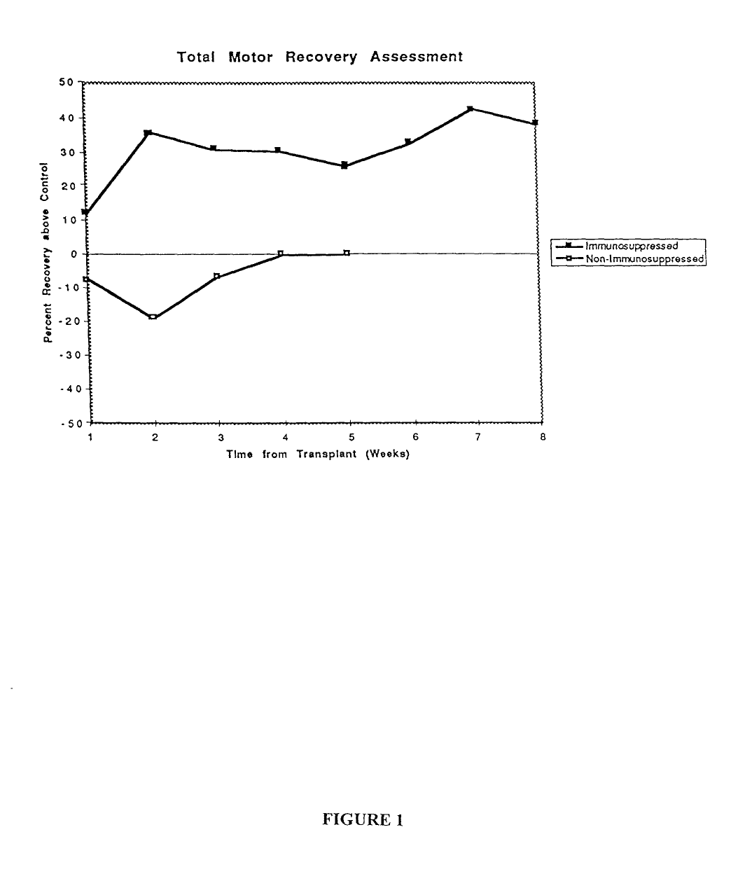

While still under anesthesia after the hemisection, rats which were to receive cellular implantations of the cellular implantation of fetal porcine cells were secured with spinal clips attached to the transverse spinous process of L2. This immobilized the spinal cord, minimized the chance of damage due to movement, and ensured reproducible positioning of the transplant in a straight dorso-ventral plane. Gelfoa™ (Upjohn) was saturated with 1×10⁶ porcine fetal spinal cord cells isolated between days 25 and 29 of gestation (n=13) or saline (n=6). The gelfoam saturated with cells or saline were grafted into the hemisected space. Following the surgical procedure, the exposed spinal cord was covered with Durafilm (Codman Surtlef) to inhibit adherence to the surrounding tissue or bone. Some of the cell transplant recipients received daily immunosuppressive therapy (cyclosporin 10 mg/kg), while others did not receive any immunosuppressive treatment. The results of transplant survival in cell recipients with or without immunosuppressive treatment are shown in FIG. 1. Immunosuppressed rats transplanted with porcine spinal cord cells survived and demonstrated increased total motor recovery up to 8 weeks post transplantation as compared to control rats which were treated with saline soaked gelfoam after hemisection.

Animals were allowed to recover from surgical procedures for a least 6 days prior to any behavioral assessment. Sensorimotor behavior was examined weekly for 8 weeks, using different tests to assess locomotor and sensory recovery.

Behavioral Motor Testing

Behavioral motor testing was used to monitor spontaneous or conditioned bilateral hindlimb functions. Rats were tested on day 7, 14, 21, 28, 35, 42, 49, and 56 postoperatively. To ensure reliability of behavioral test scores, each test was conducted at the same time of day by a blinded observer.

Multiple tests were utilized to assess improvement of sensorimotor neuron conditioned and spontaneous function. Conditioned examination involves coordinated use of both hindlimbs, while spontaneous behavior distinguishes individual hindlimb activity. The open field, righting reflex, grid walking and inclined plane test were used to determine bilateral conditioned hindlimb function. Spontaneous behavioral test included, footprint analysis and the contact placing reflex test. These behavioral tests were performed sequentially in the order of increasing difficulty. Thus, if an animal was unable to perform accordingly (receive a passing score defined under specifications of the testing parameter described below) on a test in either the conditioned or spontaneous function category, it was not subjected to the remaining behavioral exams.

I. Spontaneous Function

Contact Placing Reflex Test

The placing reflex was used to monitor the ability of both saline-treated control rats and hemisected rats receiving spinal cord cell transplants to place the injured hindlimb when the limb is brought into contact with the cage surface. The ability to place the foot was assessed for 5 trials. A score of 5 corresponded to 5 successful placements, while a score of 0 demonstrated a rat's inability to place for all 5 open trials. For comparison purposes, a control rats received a laminectomy without spinal cord injury. All rats received a score of 5 prior to surgical intervention.

In addition, to demonstrate that porcine spinal cord cells which are modified by binding anti-MHC class I antibodies to the MHC class I antigens on their surface survive and function in a xenogeneic subject, some of the spinal cord transplants were performed with porcine spinal cord cells that were masked prior to transplantation. Porcine spinal cord cells were incubated with F(ab′)₂ fragments of PT-85, a mouse monoclonal antibody specific for porcine MHC class I (i.e., “masked”). This incubation was performed in PBS for 1 hour at 4° C. with 1 μg antibody/10⁶ cells. Prior to transplantation the cells were washed in Hanks solution at 4° C. to remove unbound antibody. Additional controls included hemisected rats which received gelfoam soaked in saline and nude hemisected rats which were transplanted with unmasked porcine spinal cord cells and did not receive cyclosporin.

Testing was conducted weekly for a maximum of 8 weeks, the shortened time points in some experimental groups demonstrates that testing is currently being conducted. Surgeries consisted of either a laminectomy without spinal cord injury, or hemisection with transplantation of gelfoam saturated with fetal spinal cord porcine neurons that were either masked or unmasked (CyA treated rats) or gelfoam soaked saline. Nude hemisected rats were not immunosuppressed and transplanted with gelfoam saturated with unmasked fetal spinal cord porcine neurons.

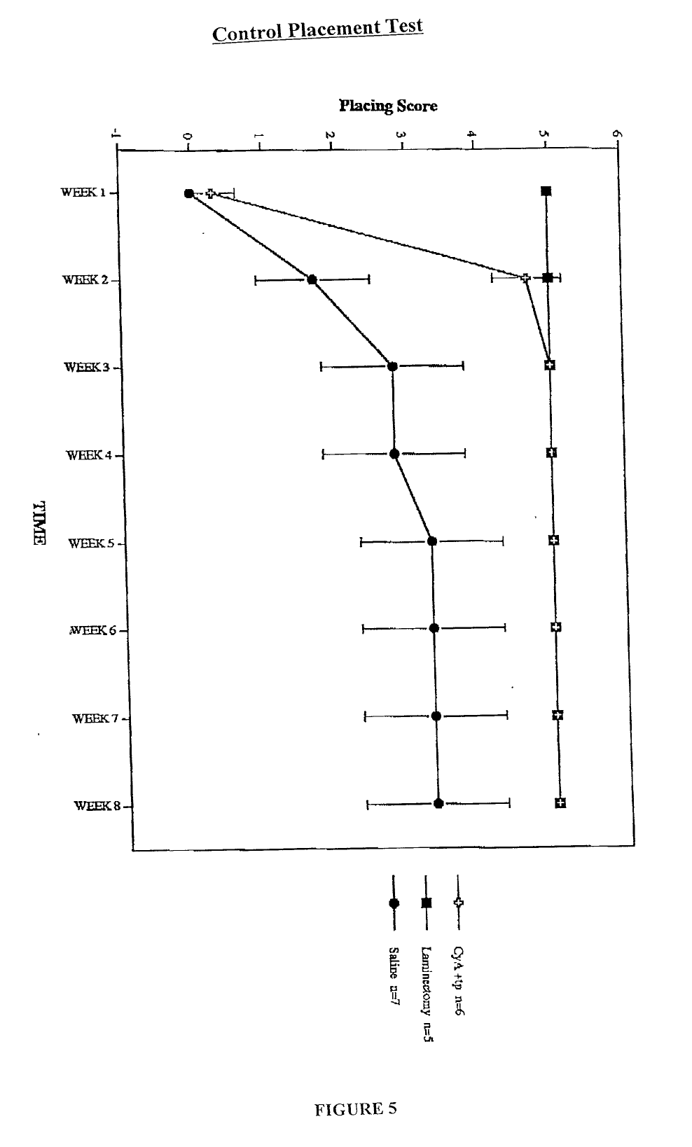

As demonstrated in FIG. 5, hemisected rats which received porcine spinal cord cells received scores as high as 5 for up to eight weeks post-transplantation, whereas, saline-treated control rats received scores only as high as 3.

II. Conditioned Function

A. Open Field

The open field test was used to determine locomotor ability by assessing the ability of hemisected rats transplanted with porcine spinal cord cells to take consistently plantar steps and to keep consistent trunk stability which walking across an open space. For comparison purposes, a control rats received a laminectomy without spinal cord injury. All rats displayed normal locomotion and received a score of 21 prior to surgical intervention.

In addition, to demonstrate that porcine spinal cord cells which are modified by binding anti-MHC class I antibodies to the MHC class I antigens on their surface survive and function in a xenogeneic subject, some of the spinal cord transplants were performed with porcine spinal cord cells that were masked prior to transplantation. Porcine spinal cord cells were incubated with F(ab′)₂ fragments of PT-85, a mouse monoclonal antibody specific for porcine MHC class I (i.e., “masked”). This incubation was performed in PBS for 1 hour at 4° C. with 1 μg antibody/10⁶ cells. Prior to transplantation the cells were washed in Hanks solution at 4° C. to remove unbound antibody. Additional controls included hemisected rats which received gelfoam soaked in saline and nude hemisected rats which were transplanted with unmasked porcine spinal cord cells and did not receive cyclosporin.

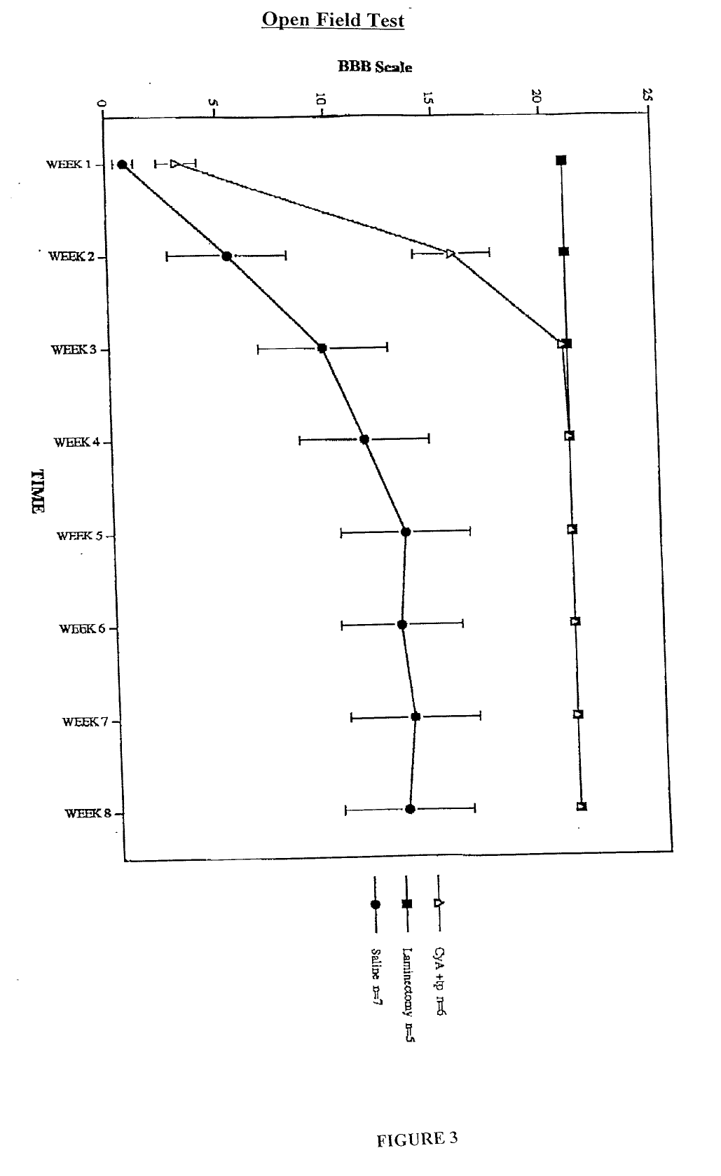

Testing was conducted weekly for a maximum of 8 weeks. As demonstrated in FIG. 3, hemisected rats treated transplanted with fetal porcine spinal cord neurons demonstrated improved locomotor ability in the open field test over extended time periods as determined by comparing to the laminectomy control rats. Moreover, as compared to the hemisected rats which were transplanted with saline, the hemisected rats receiving cellular transplants demonstrate enhance locomotor ability.

B. Inclined Plane

The inclined plane was used to determine a rat's ability to compensate behaviorally for displacement in space (Stokes et al. (1992) Exp. Neurology 116:1-12). The maximum inclination at which a rat could maintain balance (not roll off the testing apparatus) for 5 seconds was recorded. The highest angle on which balance was achieved represents the functional ability of affected hemisected spinal pathways (dorsal spinal cerebellar tract and the corticospinal tract). The angle was inclined or declined until the rat was able to maintain balance for the 5 monitored seconds. The plane was inclined to angles between 20 and 65 degrees; animals received a score corresponding to the steepest incline on which they remained balanced.

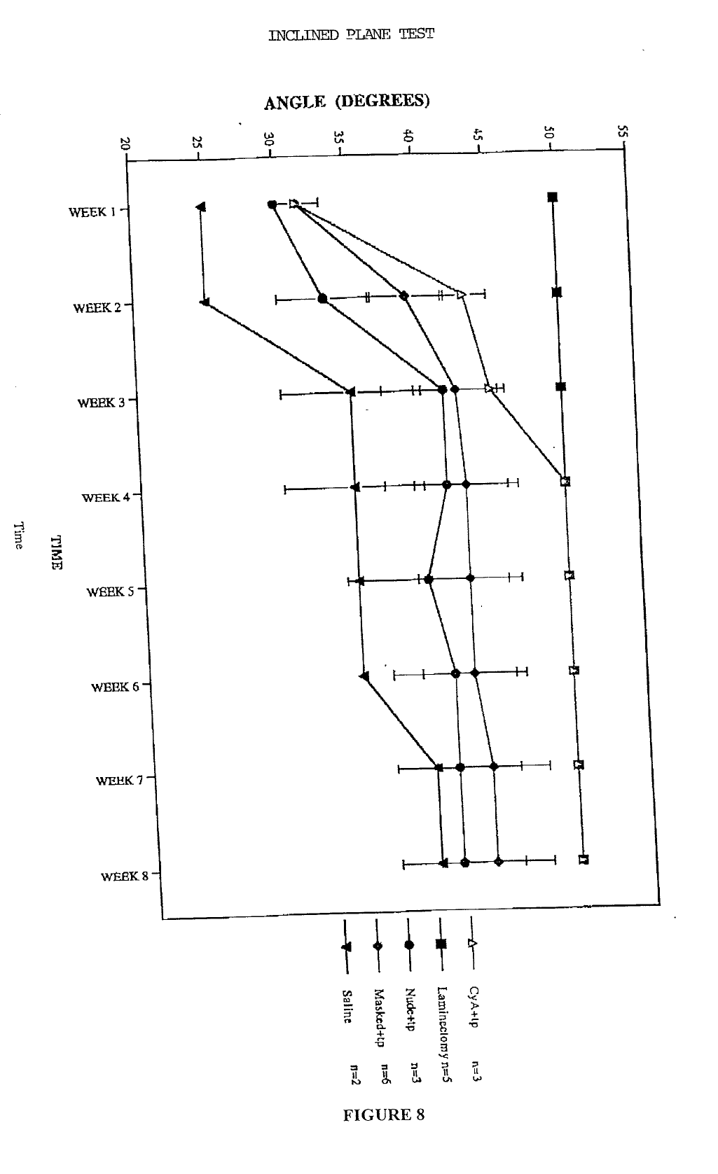

The maximum angle was 50 degrees for all animals prior to surgical intervention. Testing was conducted weekly for a maximum of 8 weeks. Surgeries consisted of either a laminectomy without spinal cord injury, or hemisection with transplantation of gelfoam saturated with fetal spinal cord porcine neurons that were either masked or unmasked (CyA treated rats) or gelfoam soaked saline. Nude hemisected rats were not immunosuppressed and transplanted with gelfoam saturated with unmasked fetal spinal cord porcine neurons. Cells were masked as described above. As shown in FIG. 8, hemisected rats which received porcine spinal cord cells were able to maintain balance between about a 40 to 50 degree incline for up to eight weeks post-transplantation, whereas, saline-treated control rats were only able to maintain balance at about a 35 to 40 degree incline.

C. Beam Walking Test

Beam walking was used to test the ability of hemisected rats with or without porcine spinal cord transplants to transverse an elevated 4.7 cm wooden beam. A score of 5 was used to denote correct placement and coordinated movement of both feet, 4 corresponded to proper foot placement of the injured hindpaw without coordinated locomotion, 3 equaled occasional foot placement and stepping, 2 corresponded to dragging the injured foot when walking on the beam, 1 denoted the ability to place the ipsilateral foot on the beam but no stepping occurred, and 0 was the inability to balance. All rats received a score of 5 prior to surgical intervention.

Testing was conducted weekly for a maximum of 8 weeks. Surgeries consisted of either a laminectomy without spinal cord injury, or hemisection with transplantation of gelfoam saturated with fetal spinal cord porcine neurons that were either masked or unmasked (CyA treated rats) or gelfoam soaked saline. Nude hemisected rats were not immunosuppressed and transplanted with gelfoam saturated with unmasked fetal spinal cord porcine neurons. Masked cells were prepared as described above. Hemisected rats which received cellular transplants received scores greater than 2 up to eight weeks post transplantation whereas saline treated rats received scores between 0 and 1.

D. Righting Reflex Test

The righting reflex was used to assesses the ability of the rat to “right” (land on all four paws instantaneously) when released 5 cm above the surface ventral side up. A score of 2 corresponded to instantaneous righting, a score of 1 was received when righting occurs, but it is not immediate, and 0 corresponded to the inability to right. All rats received a score of 2 prior to surgical intervention.

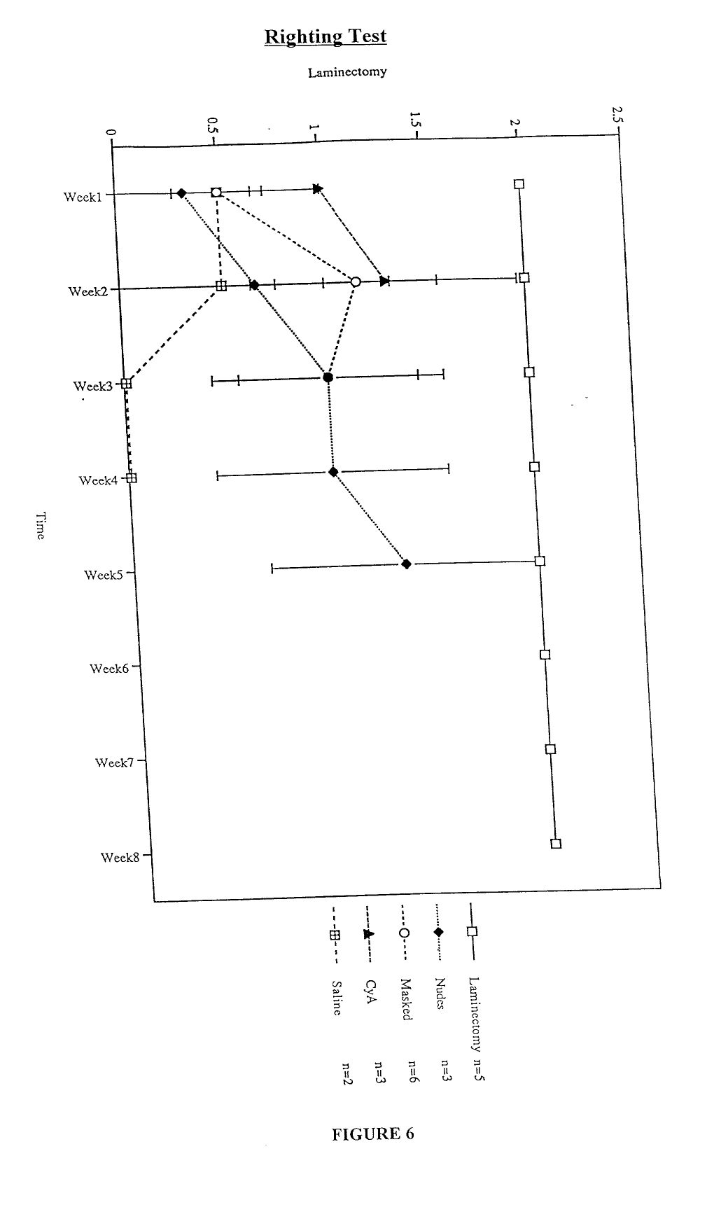

Testing was conducted weekly for a maximum of 8 weeks. Surgeries consisted of either a laminectomy without spinal cord injury, or hemisection with transplantation of gelfoam saturated with fetal spinal cord porcine neurons that were either masked or unmasked (CyA treated rats) or gelfoam soaked saline. Nude hemisected rats were not immunosuppressed and transplanted with gelfoam saturated with unmasked fetal spinal cord porcine neurons. Masked porcine spinal cord cells were prepared as described above. As shown in FIG. 6, hemisected rats which received cellular transplants received scores between 1 and 1.5 up to 5 weeks post transplantation, whereas, saline treated rats received scores between 0 and 0.5.

E. Grasping Reflex

The grasping reflex was used to monitor the ability of both saline-treated rats and rats receiving spinal cord cell transplants to grasp a wire mesh screen (60 cm×45 cm). The ability to grasp onto 5 open squares (1 cm×l cm) was assessed. A score of 5 corresponded to 5 successful grasps, while a score of 0 demonstrated a rat's inability to grasp while transversing all 5 open squares. All rats received a score of 5 prior to surgical intervention.

Testing was conducted weekly for a maximum of 8 weeks, the shortened time points in some experimental groups demonstrates that testing is currently being conducted. Surgeries consisted of either a laminectomy without spinal cord injury, or hemisection with transplantation of gelfoam saturated with fetal spinal cord porcine neurons that were either masked or unmasked (CyA treated rats) or gelfoam soaked saline. Nude hemisected rats were not immunosuppressed and transplanted with gelfoam saturated with unmasked fetal spinal cord porcine neurons.

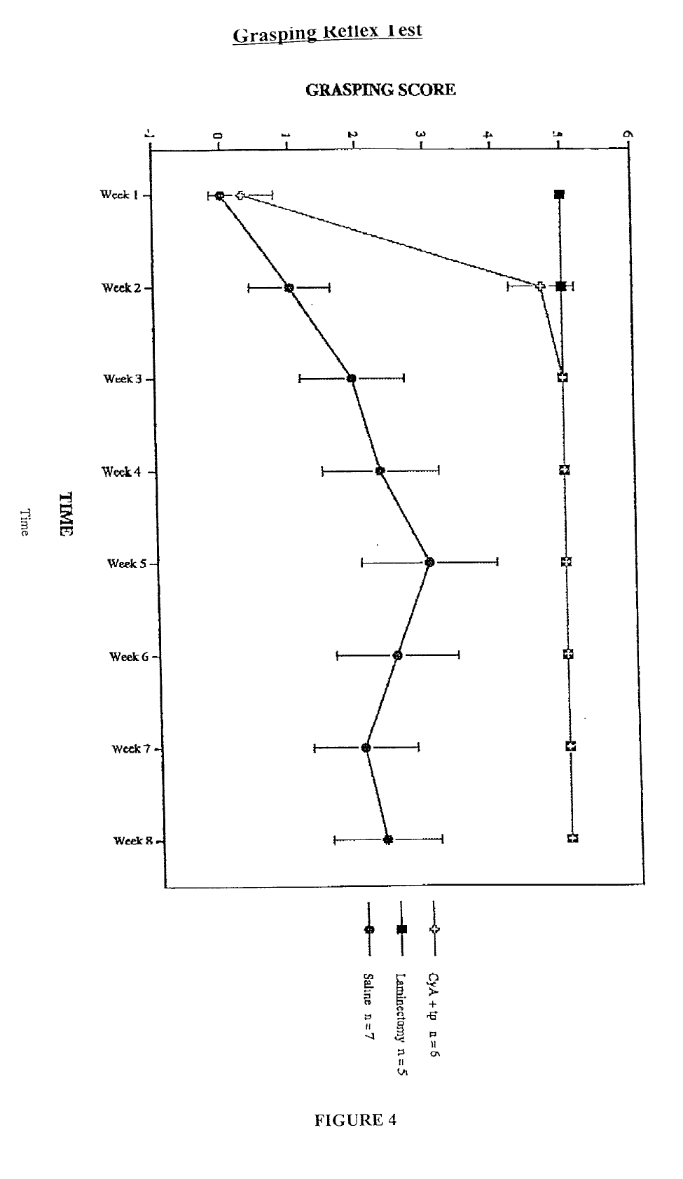

As demonstrated in FIG. 4, hemisected rats which received cellular transplants received scores as high as 5 up to eight weeks post transplantation, whereas, saline treated rats received scores received scores of 0 to 3.

Analysis of Locomotor Behavior

Locomotor behavior was analyzed by first calculating the net improvement score for the experimental treatment by subtracting the mean score for spontaneous recovery (animals with injury only). This value was then converted to a percent of maximal recovery by dividing the mean value by the maximum response defined by each specific test (maximum scores of 21 for the open field test, 5 for grid walking, and 5 for contact placing). In this way, the value represented a true increase in motor score due to the experimental treatment. In addition, the improvement of control animals which were hemisected and then treated with cyclosporin alone were analyzed and plotted in the same manner, thus providing a direct comparison between cyclosporin-dependent recovery and cell-specific effects.

The combined scores from the behavioral tests demonstrated 40% greater locomotor recovery in the fetal porcine grafted rats compared to controls within 2 to 3 weeks after transplantation. The enhanced locomotor performance observed suggests that transplantation shows promise as a novel treatment for individuals suffering from spinal cord damage.

In addition, the results from the control rats demonstrate that rats treated with cyclosporin without transplantation of fetal porcine neurons did not show enhanced recovery of locomotor functions. These experiments were used to eliminate the possibilities of spontaneous recovery and effects of the immunosuppressive therapy upon enhancing functional recovery. The results suggest that the observed functional recovery in SCI rats may be attributed solely to the transplanted neurons.

III. Sensory Abnormally Test

The abnormal sensation of allodynia (innocuous stimuli perceived as painful) was determined by using von Frey thresholds. Tactile allodynia were assessed with a series of calibrated von Frey hairs (Stoleting, Forest Hills, Ill.) applied to the glabrous hind paw surface until hair begins to bend. This process was repeated 5 times at a frequency of approximately 2/sec (Seltzer, 1991) or until the paw lifted. A score was given according the smallest force (hair diameter) which causes a lifting response. Normal animals respond at 75.7 g of force whereas allodynic animals responded at lower forces. Testing was alternated between both hind paws until a withdrawal threshold was reached (average of both sides).

Histological Assessment of Porcine Graft Survival and Integration

In order to determine the level of fetal porcine neuron integration, survival, and sprouting, various histological markers were applied to perfused rat spinal cords following conclusion of all behavioral tests. The animals were transcardially perfused and post fixed with 4% parafornaldehyde. Tissue sections of spinal cord segments at the graft site as well as adjacent tissue approximately 14 mm from the graft site were cut on a freezing microtome or cryostat. Representative sections were stained with Nissl or hematoxylin and eosin to characterize cell morphology in the injured area. Sections were analyzed for the integration of the graft, as well as, the degree and organization of repaired or regenerated spinal tracts. These experiments allowed for identification the extent of synaptic integration with the host by measuring supraspinal serotonergic (5-HT) innervation and axonal projections from the dorsal root ganglia using calcitonin gene-related peptide (CGRP) as a marker. The 5-HT immunostain was applied as a marker to demonstrate the transplant-mediated reestablishment of host spinal tracts on the cellular level, while the behavioral data demonstrated the beneficial effects of the grafts at the functional anatomical level. The plasticity of the spinal cord in spinal cord injury fetal transplanted rats was determined by labeling host projections with CGRP. CGRP demonstrates that host neurons are regenerated by staining for CGRP in spinal segments adjacent to the injury, and whether these projections are able to establish synaptic connections, as determined by behavioral tests.

Histological analysis detected porcine positive neurons at the site of implantation (n=8), in addition, there was a restoration of CGRP and 5-HT staining in adjacent spinal sections (n=8).

### Example III

### Transplantation of Porcine Spinal Cord Cells into Mouse Model for ALS

Fetal porcine spinal cord cells were isolated as described above and injected into SOD mice (commercially available from Jackson Labs) which are therapeutically predictive of results in humans with ALS. Transplanted mice all received 100,000 fetal pig spinal cord cells (E26-28) to one side of the spine at lumbar level L1. Cell placement was in the ventral horn of the spinal cord where motor neurons are located. All animals were administered systemic immunosuppression of 50 mg/kg cyclosporin A, subcutaneous, daily.

Symptomatic testing was not started until about two and half months after the birth of the SOD mice when all but one mouse showed signs of being affected.

As demonstrated in Table I, within about twenty days after the onset of symptoms, all of the SOD mice injected with saline had died (i.e., animal numbers: 330-2D, 330-2T, 330-2U), whereas the SOD mice transplanted with porcine spinal cord cells (animal numbers: 330-2G and 330-2H), have survived up to 2 months after the onset of symptoms.

TABLE I

| animal | | | Transplant date | | Symptom | |
|---|---|---|---|---|---|---|
| no. # | genotype | D.O.B. | & Type | | onset date | Died |
| 330-2 E | (−) | 2/24 | 5/5: | saline | | |
| 330-2 F | (−) | 2/24 | 5/5: | saline | | |
| 330-2 D | (+) | 2/24 | 5/5: | saline | 5/11 | 5/26 |
| 330-2 T | (+) | 2/24 | 5/5: | saline | 5/13 | 5/23 |
| 330-2 U | (+) | 2/24 | 5/5: | saline | 5/11 | 5/22 |
| 330-2 S | (+) | 2/24 | 5/5: | cells | | 5/5: |
| | | | | | | anesthesia |
| 330-2 G | (+) | 2/24 | 5/5: | cells | 5/11 | |
| 330-2 H | (+) | 2/24 | 5/5: | cells | 5/11 | |

### Example IV

### Methods of Detecting Pathogens in Swine

A. Collecting, Processing, and Analyzing Pig Fecal Samples for Signs of Pathogens

Feces are extracted from the pig's rectum manually and placed in a sterile container. About a 1.5 cm diameter portion of the specimen was mixed thoroughly in 10 ml of 0.85% saline. The mixture is then strained slowly through a wire mesh strainer into a 15 ml conical centrifuge tube and centrifuged at 650×g for 2 minutes to sediment the remaining fecal material. The supernatant is decanted carefully so as not to dislodge the sediment and 10% buffered formalin was added to the 9 ml mark, followed by thorough mixing. The mixture is allowed to stand for 5 minutes. 4 ml of ethyl acetate is added to the mixture and the mixture is capped and mixed vigorously in an inverted position for 30 seconds. The cap is then removed to allow for ventilation and then replaced. The mixture is centrifuged at 500×g for 1 minute (four layers should result: ethyl acetate, debris plug, formalin and sediment). The debris plug is rimmed using an applicator stick. The top three layers are carefully discarded by pouring them off into a solvent container. The debris attached to the sides of the tube is removed using a cotton applicator swab. The sediment is mixed in either a drop of formalin or the small amount of formalin which remains in the tube after decanting. Two separate drops are placed on a slide to which a drop of Lugol's iodine is added. Both drops are coverslipped and carefully examined for signs of pathogens, e.g., protozoan cysts of trophozoites, helminth eggs and larvae. Protozoan cyst identification is confirmed, when required, by trichrome staining.

B. Co-cultivation Assay for Detecting the Presence of Human and Animal Viruses in Pig Cells

Materials

Cell Lines

African green monkey kidney, (VERO), cell line American Type Culture Collection, (ATCC CCL8 1), human embryonic lung fibroblasts, (MRC-5) cell line American Type Culture Collection, (ATCC CCL 171), porcine kidney, (PK-15), cell line American Type Culture Collection, (ATCC CRL 33), porcine fetal testis, (ST), cell line American Type Culture Collection, (ATCC CRL 1746)

Medium, Antibiotics, and Other Cells, and Equipment

Fetal calf serum, DMEM, Penicillin 10,000 units/ml, Streptomycin 10 mg/ml, Gentamicin 50 mg/ml, guinea pig erythrocytes, chicken erythrocytes, porcine erythrocytes, Negative Control (sterile cell culture medium), Positive Controls: VERO and MRC-5 Cells: Poliovirus type 1 attenuated, (ATCC VR-1 92) and Measles virus, Edmonston strain, (ATCC VR-24), PK-1 5 and ST Cells: Swine influenza type A, (ATCC VR-99), Porcine Parvovirus, (ATCC VR-742), and Transmissible gastroenteritis of swine, (ATCC VR-743). Equipment: tissue Culture Incubator, Inverted Microscope, Biological Safety Cabinet.

These materials can be used in a co-cultivation assay (a process whereby a test article is inoculated into cell lines (VERO, MRC-5, PK1 5, and ST) capable of detecting a broad range of human, porcine and other animal viruses). Hsuing, G. D., “Points to Consider in the Characterization of Cell Lines Used to Produce Biologicals” in Diagnostic Virology, 1982 (Yale University Press, New Haven, Conn., 1982).

Experimental Design and Methodology

A total of three flasks (T25) of each cell line are inoculated with at least 1 ml of test article. Three flasks of each cell line can also be inoculated with the appropriate sterile cell culture medium as a negative control. Positive control viruses are inoculated into three flasks of each cell line. After an absorption period, the inoculate is removed and all flasks incubated at 35-37° C. for 21 days. All flasks are observed at least three times per week for the development of cytopathic effects, (CPE), of viral origin. Harvests are made from any flasks inoculated with the test article that show viral CPE.

At Day 7 an aliquot of supernatant and cells from the flasks of each test article are collected and at least 1 ml is inoculated into each of three new flasks of each cell line. These subcultures are incubated at 35-37° C. for at least 14 days. All flasks are observed and tested as described above.

At Day 7, the flasks from each test article are also tested for viral hemadsorption, (HAd), using guinea pig, monkey and chicken erythrocytes at 2-8° C. and 35-37° C. at 14 days postinoculation.

At Day 21, if no CPE is noted, an aliquot of supernatant from each flask is collected, pooled, and tested for viral hemagglutination, (HA), using guinea pig, monkey, and chicken erythrocytes at 2-8° C. and 35-37° C. Viral identification is based on characteristic viral cytopathic effects (CPE) and reactivity in HA HAd testing.

The test samples are observed for viral cytopathic effects in the following manner: All cultures are observed for viral CPE at least three times each week for a minimum of 21 days incubation. Cultures are removed from the incubator and observed using an inverted microscope using at least 40×magnification. 100× or 200×magnification is used as appropriate. If any abnormalities in the cell monolayers, including viral CPE, are noted or any test articles cause total destruction of the cell monolayer, supernatant and cells are collected from the flasks and samples are subcultured in additional flasks of the same cell line. Samples can be stored at −60° to −80° C. until subcultured. After 7 and 14 days incubation, two blind passages are made of each test article by collecting supernatant and cells from all flasks inoculated with each sample. Samples can be stored at −60° to −80° C. until subcultured.

Hemadsorbing viruses are detected by the following procedure: after 21 days of incubation, a hemadsorption test is performed to detect the presence of hemadsorbing viruses. Supernatant fluids are collected and pooled from each flask inoculated with test articles or controls. Fluids are tested using guinea pig, monkey, and chicken erythrocytes. Hemagglutination testing is also performed after 21 days of incubation of the subcultures. Viral isolates are identified based on the cell line where growth was noted, the characteristics of the viral CPE, the hemadsorption reaction, and hemagglutination reactions, as appropriate. The test article is considered negative for the presence of a viral agent, if any of the cell lines used in the study demonstrate viral, CPE, HA, or HAd in a valid assay.

C. Procedure for Preparing and Maintaining Cell Lines Used to Detect Viruses in Pig Cells

Materials

Fetal calf serum (FCS), DMEM, Penicillin 10,000 unit/ml, Streptomycin 10 mg/ml, Gentamicin 50 mg/ml, T25 tissue culture flasks, tissue culture incubator (5% CO₂, 37° C.)

Procedure

Aseptic techniques are followed when performing inoculations and transfers. All inoculations and transfers are performed in a biological safety cabinet. Media is prepared by adding 10% FCS for initial seeding, 5% FCS for maintenance of cultures, as well as 5.0 ml of penicillin/streptomycin and 0.5 ml of gentamicin per 500 ml media. Sufficient media is added to cover the bottom of a T25 tissue culture flask. The flask is seeded with the desired cell line and incubated at 37° C., 5% CO₂ until cells are 80 to 100% confluent. The flasks are then inoculated with virus (QCP25).

D. Preparation of Erythrocyte (rbc) Suspensions Used in Hemadsorption (HAd) and Hemagglutination (HA) Virus Detection Testing

Materials

Phosphate buffered saline, (PBS), pH 7.2, guinea pig erythrocytes stock solution, porcine erythrocytes stock solution, chicken erythrocytes stock solution, sterile, disposable centrifuge tubes, 15 or 50 ml Laboratory centrifuge

Procedure

An appropriate amount of erythrocytes (rbc) is obtained from stock solution. The erythrocytes are washed 3 times with PBS by centrifugation at approximately 1000×g for 10 minutes. A 10% suspension is prepared by adding 9 parts of PBS to each one part of packed erythrocytes. The 10% rcb suspensions are stored at 2-8° C. for no more than one week. 0.5% ecb suspensions are prepared by adding 19 parts of PBS to each one part of 10% rbc suspension. Fresh 0.5% rbc suspensions are prepared prior to each day's testing.

Hemagglutination (HA) Test

A hemagglutination test is a test that detects viruses with the property to agglutinate erythrocytes, such as swine influenza type A, parainfluenza, and encephalomyocarditis viruses, in the test article. Hsuing, G. D. (1982) Diagnostic Virology (Yale University Press, New Haven, Conn.);. Stites, Daniel P and Terr, Abba I, (1991), Basic and Clinical Immunology (Appleton & Lange, East Norwalk, Conn.).

Materials

Supernatants from flasks of the VERO cell line, MRC-5 inoculated with the test article, flasks of positive and negative controls, phosphate buffered saline (PBS), pH 7.2, guinea pig erythrocytes (GPRBC), 0.5% suspension in PBS, chicken erythrocytes (CRBC), 0.5% suspension in PBS, porcine erythrocytes (MRBC), 0.5% suspension in PBS.

Procedure

All sample collection and testing is performed in an approved biological safety cabinet. 0.5% suspensions of each type of erythrocytes are prepared as described above. The HA test on all cell lines inoculated with samples of the test articles at least 14 days post-inoculation. Positive and negative control cultures are included for each sample and monolayers are examined to ensure that they are intact prior to collecting samples.

At least 1 ml of culture fluid from each flask inoculated with the test article is collected and pooled. 1 ml samples from the negative and positive control cultures are also collected and pooled. A set of tubes is labeled with the sample number and type of erythrocyte (distinguish positive and negative suspension) to be added. Racks may be labeled to differentiate the type of erythrocyte. 0.1 ml of sample is added to each tube. 0.1 ml of the appropriate erythrocyte suspension is added to each tube. Each tube is covered with parafilm and mixed thoroughly. One set of tubes is incubated at 2-8° C. until tight buttons form in the negative control in about 30-60 minutes. Another set of tubes is incubated at 35-37° C. until tight buttons form in the negative control in about 30-60 minutes.

Formation of a tight button of erythrocytes indicates a negative result. A coating of the bottom of the tube with the erythrocytes indicates a positive result.

E. Methods Used for Fluorescent Antibody Stain of Cell Suspensions Obtained from Flasks Used in Detection of Viruses in Porcine Cells Using Cell Culture Techniques (as Described in Sections B and C)

Materials

Pseudorabies, parvovirus, enterovirus. adenovirus, transmissible Gastroenteritis Virus. bovine viral diarrhea, encephalomyocarditis virus, parainfluenza, vesicular stomatitis virus., microscope slides, PBS, incubator with humidifying chamber at 36° C., Evan's blue coutner stain, DI Water, fluorescent microscope, trypsin, serum containing media, acetone, T25 Flask.

Procedure

Cells (described in Sections B and C) are trypsinized to detach them from the T25 flask and sufficient media is added to neutralize trypsin activity. A drop of cell suspension is placed on each microscope slide and allowed to air dry. A slide for each fluorescent antibody is prepared. Cells are fixed by immersion in acetone for five minutes. Each fluorescent antibody solution is placed on each slide to cover cells and the slides are incubated in humidifying chamber in incubator at 36° C. for 30 minutes. The slides are then washed in PBS for five minutes. The wash is repeated in fresh PBS for five minutes followed by a rinse with DI water.

The cells are counterstained by placing Evan's blue solution on each slide to cover cells for five minutes at room temperature. The slides are then washed in PBS for five minutes. The wash is repeated in fresh PBS for five minutes followed by a rinse with DI water. The slides are then allowed to air dry. Each slide is inspected under a fluorescent microscope. Any fluorescent inclusion bodies characteristic of infection are considered a positive result for the presence of virus.

F. Procedures for Defining Bacteremic Pigs

Materials

Anaerobic BMB agar (5% sheep blood, vitamin K and hemin [BMB/blood]), chocolate Agar with Iso Vitalex, Sabaroud dextrose agar/Emmons, 70% isopropyl alcohol swabs, betadine solution, 5% CO₂ incubator at 35-37° C., anaerobic blood agar plate, gram stain reagents (Columbia Broth Media), aerobic blood culture media (anaerobic brain heart infusion with vitamin K& hemin), septicheck media system, vitek bacterial identification system, laminar flow hood, microscope, and bacteroids and Bacillus stocks.

Procedure

Under a laminar flow hood, disinfect the tops of bottles for aerobic and anaerobic blood cultures of blood obtained from pig with 70% isopropyl alcohol, then with betadine The rubber stopper and cap from the aerobic blood culture bottle are removed and a renal septicheck media system is attached to the bottle. The bottles are incubated in 5% CO₂ for 21 days at 35-37° C., and observed daily for any signs of bacterial growth (i.e. gas bubbles, turbidity, discoloration or discrete clumps). Negative controls consisting of 5 cc of sterile saline in each bottle and positive controls consisting of Bacillus subtilis in the aerobic bottle and Bacteriodes Vulgaris in the anaerobic bottle are used. If signs of bacterial growth are observed, a Gram stain is prepared and viewed microscopically at 100× oil immersion for the presence of any bacteria or fungi. The positive bottles are then subcultured onto both chocolate agar plates with Iso Vitlex and onto BMB plates. The chocolate plate is incubated at 35-37° C. in 5% CO₂ for 24 hours and the BMB anaerobically at 35-37° C. for 48 yeast or fungi that is in evidence at gram stain is subcultured onto a Sabaroud dextrose/Emmons plate. The Vitek automated system is used to identify bacteria and yeast. Fungi are identified via their macroscopic and microscopic characteristic. If no signs of growth are observed at the end of 21 days, gram stain is prepared and observed microscopically for the presence of bacteria and fungi.

Absence of growth in the negative control bottles and presence of growth in the positive control bottles indicates a valid test. The absence of any signs of growth in both the aerobic and anaerobic blood culture bottles, as well as no organisms seen on gram stain indicates a negative blood culture. The presence and identification of microorganism(s) in either the aerobic or anaerobic blood culture bottle indicates of a positive blood culture; this typicall is due to a bacteremic state.

1. A composition for transplantation into a xenogeneic subject comprising an isolated spinal cord cell obtained from a pig, such that treatment of spinal cord damage is obtained upon transplantation into the subject.

2. The composition of claim 1, wherein the pig is an embryonic pig.

3. The composition of claim 2, wherein the spinal cord cell is isolated from an embryonic pig between about days 20 to 30 of gestation.

4. The composition of claim 3, wherein the spinal cord cell is isolated from an embryonic pig between about days 25 to 29 of gestation.

5. The composition of claim 1, wherein the spinal cord cell is an oligodendrocyte.

6. The composition of claim 1, wherein the spinal cord cell is an astrocyte.

7. The composition of claim 1, wherein the spinal cord cell is a neuron.

8. The composition of claim 1, wherein the cell, in unmodified form, has an antigen on the cell surface which is capable of stimulating an immune response against the cell in a xenogeneic subject, wherein the antigen on the cell surface is altered to inhibit rejection of the cell upon introduction of the composition into the subject.

9. The composition of claim 8, wherein the antigen on the cell surface which is altered is an MHC class I antigen.

10. The composition of claim 9, wherein the cell is contacted prior to transplantation into the human with at least one anti-MHC class I antibody or fragment thereof, which binds to the MHC class I antigen on the cell surface but does not activate complement or induce lysis of the cell.

11. The composition of claim 10, wherein the anti-MHC class I antibody is an anti-MHC class I F(ab′)₂ fragment.

12. The composition of claim 11, wherein the anti-MHC class I F(ab′)₂ fragment is a F(ab′)₂ fragment of a monoclonal antibody PT85.

13. The composition of claim 1, which further comprises at least one of the agents or factors selected from the group consisting of neurotrophic factors and anti-inflammatory agents.

14. The composition of claim 13, wherein the neurotrophic factor is selected from the group consisting of brain-derived neurotrophic factor, platelet-derived neurotrophic factor, neural growth factor, ciliary neurotrophic factor, neurotrophin-3, neurotrophin 4/5 and basic fibroblast growth factor.

15. The composition of claim 13, wherein the anti-inflammatory agent is a steroid.

16. The composition of claim 15, wherein the steroid is methylprednisolone.

17. The composition of claim 1, wherein the cell is obtained from a pig predetermined to be free from at least one organism selected from the group consisting of zoonotic, cross-placental and neurotropic organisms.

18. A method of treating a xenogeneic subject having spinal cord damage by administering to the subject a composition comprising an isolated spinal cord cell obtained from a pig, such that treatment of spinal cord damage is obtained upon administration of the composition to the subject.

19. The method of claim 18, wherein the spinal cord cell is obtained from an embryonic pig.

20. The method of claim 19, wherein the spinal cord cell is isolated from an embryonic pig between about days 20 to 30 of gestation.

21. The method of claim 20, wherein the spinal cord cell is isolated from an embryonic pig between about days 25 to 29 of gestation.

22. The method of claim 18, wherein the spinal cord cell is an oligodendrocyte.

23. The method of claim 18, wherein the spinal cord cell is an astrocyte.

24. The method of claim 18, wherein the spinal cord cell is a neuron.

25. The method of claim 18, wherein the cell, in unmodified form, has at least one antigen on the cell surface which is capable of stimulating an immune response against the cells in the subject, wherein the antigen on the cell surface is altered to inhibit rejection of the cells when introduced into the subject.

26. The method of claim 25, wherein the cell is contacted prior to introduction into the subject with at least one molecule which binds to at least one antigen on the cell surface which is capable of stimulating an immune response against the cell in the subject to alter the antigen on the cell surface to inhibit rejection of the cell when introduced into the subject.

27. The method of claim 26, wherein the antigen on the surface of the cell which is altered is an MHC class I antigen.

28. The method of claim 26, wherein the cell is contacted prior to introduction into the subject with at least one anti-MHC class I antibody or fragment thereof, which binds to the MHC class I antigen on the cell surface but does not activate complement or induce lysis of the cell.

29. The method of claim 28, wherein the anti-MHC class I antibody is an anti-MHC class I F(ab′)₂ fragment.

30. The method of claim 29, wherein the anti-MHC class I F(ab′)₂ fragment is a F(ab′)₂ fragment of a monoclonal antibody PT85.

31. The method of claim 18, wherein the composition further comprises at least one of the agents or factors selected from the group consisting of neurotrophic factors and anti-inflammatory agents.

32. The method of claim 31, wherein the neurotrophic factor is selected from the group consisting of brain-derived neurotrophic factor, ciliary neurotrophic factor, platelet-derived growth factor, neural growth factor, neurotrophin-3, neurotrophin 4/5 and basic fibroblast growth factor.

33. The method of claim 31, wherein the anti-inflammatory agent is a steroid.

34. The method of claim 33, wherein the steroid is methylprednisolone.

35. The method of claim 18, wherein the xenogeneic subject is a human.

36. The method of claim 35, wherein spinal cord damage is spinal cord injury.

37. The method of claim 35, wherein the spinal cord damage is a neurodegenerative disorder.

38. The method of claim 37, wherein the neurodegenerative disorder is amyotrophic lateral sclerosis.
